SEQUENCE LISTING (15 sequences)

SEQ ID NO: 1 - protein, 766 aa, Homo sapiens
MAGCIPEEKTYRRFLELFLGEFRGPCGGGEPEPEPEPEPEPEPEPESEPEPEPELVEAEAAEASVEEPGEEAATVAATEEGDQEQDPEPEEEAAVEGEEEEEGAATAAAAPGHSAVPPPPPQLPPLPPLPRPLSERITPRPLSERITREEVEGESLDLCLQQLYKYNCPSFLAAALARATSDEVLQSDLSAHYIPKETDGTEGTVEIETVKLARSVFSKLHEICCSWVKDFPLRRRPQLYYETSIHAIKNMRRKMEDKHVCIPDFNMLFNLEDQEEQAYFAVFDGHGGVDAAIYASIHLHVNLVRQEMFPHDPAEALCRAFRVTDERFVQKAARESLRCGTTGVVTFIRGNMLHVAWVGDSQVMLVRKGQAVELMKPHKPDREDEKQRIEALGGCIVWFGAWRVNGSLSVSRAIGDAEHKPYICGDADSASTVLDGTEDYLILACDGFYDTVNPDEAVKVVSDHLKENNGDSSMVAHKLVASARDAGSSDNITVIVVFLRDMNKAVNVSEESDWTENSFQGGQEDGGDDKENHGECKRPWPQHQCSAPADLGYDGRVDSFTDRTSLSPGSQINVLEDPGYLDLTQIEASKPHSAQFLLPVEMFGPGAPKKANLINELMMEKKSVQSSLPEWSGAGEFPTAFNLGSTGEQIYRMQSLSPVCSGLENEQFKSPGNRVSRLSHLRHHYSKKWHRFRFNPKFYSFLSAQEPSHKIGTSLSSLTGSGKRNRIRSSLPWRQNSWKGYSENMRKLRKTHDIPCPDLPWSYKIE

SEQ ID NO: 2 - protein, 755 aa, Homo sapiens
MAGCIPEEKTYRRFLELFLGEFRGPCGGGEPEPEPEPEPEPEPESEPEPEPELVEAEAAEASVEEPGEEAATVAATEEGDQEQDPEPEEEAAVEGEEEEEGAATAAAAPGHSAVPPPPPQLPPLPPLPRPLSERITREEVEGESLDLCLQQLYKYNCPSFLAAALARATSDEVLQSDLSAHYIPKETDGTEGTVEIETVKLARSVFSKLHEICCSWVKDFPLRRRPQLYYETSIHAIKNMRRKMEDKHVCIPDFNMLFNLEDQEEQAYFAVFDGHGGVDAAIYASIHLHVNLVRQEMFPHDPAEALCRAFRVTDERFVQKAARESLRCGTTGVVTFIRGNMLHVAWVGDSQVMLVRKGQAVELMKPHKPDREDEKQRIEALGGCVVWFGAWRVNGSLSVSRAIGDAEHKPYICGDADSASTVLDGTEDYLILACDGFYDTVNPDEAVKVVSDHLKENNGDSSMVAHKLVASARDAGSSDNITVIVVFLRDMNKAVNVSEESDWTENSFQGGQEDGGDDKENHGECKRPWPQHQCSAPADLGYDGRVDSFTDRTSLSPGSQINVLEDPGYLDLTQIEASKPHSAQFLLPVEMFGPGAPKKANLINELMMEKKSVQSSLPEWSGAGEFPTAFNLGSTGEQIYRMQSLSPVCSGLENEQFKSPGNRVSRLSHLRHHYSKKWHRFRFNPKFYSFLSAQEPSHKIGTSLSSLTGSGKRNRIRSSLPWRQNSWKGYSENMRKLRKTHDIPCPDLPWSYKIE

SEQ ID NO: 3 - protein, 454 aa, Homo sapiens
MSSGAPQKSSPMASGAEETPGFLDTLLQDFPALLNPEDPLPWKAPGTVLSQEEVEGELAELAMGFLGSRKAPPPLAAALAHEAVSQLLQTDLSEFRKLPREEEEEEEDDDEEEKAPVTLLDAQSLAQSFFNRLWEVAGQWQKQVPLAARASQRQWLVSIHAIRNTRRKMEDRHVSLPSFNQLFGLSDPVNRAYFAVFDGHGGVDAARYAAVHVHTNAARQPELPTDPEGALREAFRRTDQMFLRKAKRERLQSGTTGVCALIAGATLHVAWLGDSQVILVQQGQVVKLMEPHRPERQDEKARIEALGGFVSHMDCWRVNGTLAVSRAIGDVFQKPYVSGEADAASRALTGSEDYLLLACDGFFDVVPHQEVVGLVQSHLTRQQGSGLRVAEELVAAARERGSHDNITVMVVFLRDPQELLEGGNQGEGDPQAEGRRQDLPSSLPEPETQAPPRS

SEQ ID NO: 4 - protein, 31 aa, Homo sapiens
MAGCIPEEKTYRRFLELFLGEFRGPCGGGEP

SEQ ID NO: 5 - protein, 453 aa, Homo sapiens
MSSGAPQKSSPMASGAEETPGFLDTLLQDFPALLNPEDPLPWKAPGTVLSQEEVEGELAELAMGFLGSRKAPPPLAAALAHEAVSQLLQTDLSEFRKLPREEEEEEEDDDEEKAPVTLLDAQSLAQSFFNRLWEVAGQWQKQVPLAARASQRQWLVSIHAIRNTRRKMEDRHVSLPSFNQLFGLSDPVNRAYFAVFDGHGGVDAARYAAVHVHTNAARQPELPTDPEGALREAFRRTDQMFLRKAKRERLQSGTTGVCALIAGATLHVAWLGDSQVILVQQGQVVKLMEPHRPERQDEKARIEALGGFVSHMDCWRVNGTLAVSRAIGDVFQKPYVSGEADAASRALTGSEDYLLLACDGFFDVVPHQEVVGLVQSHLTRQQGSGLRVAEELVAAARERGSHDNITVMVVFLRDPQELLEGGNQGEGDPQAEGRRQDLPSSLPEPETQAPPRS

SEQ ID NO: 6 - protein, 300 aa, Homo sapiens
MSSGAPQKSSPMASGAEETPGFLDTLLQDFPALLNPEDPLPWKAPGTVLSQEEVEGELAELAMGFLGSRKAPPPLAAALAHEAVSQLLQTDLSEFRKLPREEEEEEEDDDEEEKAPVTLLDAQSLAQSFFNRLWEVAGQWQKQVPLAARASQRQWLVSIHAIRNTRRKMEDRHVSLPSFNQLFGLSDPVNRAYFAVFDGHGGVDAARYAAVHVHTNAARQPELPTDPEGALREAFRRTDQMFLRKAKRERLQSGTTGVCALIAGATLHVAWLGDSQVILVQQGQVVKLMEPHRPERQDEK

SEQ ID NO: 7 - protein, 264 aa, Artificial
  GST-peptide substrate
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSGGGGRRATVAGGGGRRATVAGGGGRRATVAGGG

SEQ ID NO: 8 - protein, 14 aa, Artificial
  antigen
KTHDIPCPDLPWSY

SEQ ID NO: 9 - protein, 16 aa, Artificial
  a1antigen
CTSPPDSFLDDHHLTR

SEQ ID NO: 10 - protein, 16 aa, Artificial
  a2antigen
CMDDSAMHIPPGLKPH

SEQ ID NO: 11 - protein, 14 aa, Artificial
  ppm1a1antigen
KNDDTDSTSTDDMW

SEQ ID NO: 12 - protein, 15 aa, Artificial
  ilkapag
KAVQRGSADNVTVMV

SEQ ID NO: 13 - protein, 12 aa, Artificial
  ppm1eag
RSSLPWRQNSWK

SEQ ID NO: 14 - protein, 16 aa, Artificial
  ppm1fag
LPSSLPEPETQAPPRS

SEQ ID NO: 15 - protein, 14 aa, Artificial
  ppm1kag
FSFSRSFASSGRWA

(19) United States
(12) Patent Application Publication
(10) Pub. No.: US 2010/0069502 A1
(43) Pub. Date: Mar. 18, 2010

(54) Assay

(76) Inventor: Patricia Townsend Wade Cohen, London (GB)

Correspondence Address: BRADLEY ARANT BOULT CUMMINGS LLP;INTELLECTUAL PROPERTY DEPARTMENT, 1819 FIFTH AVENUE NORTH, BIRMINGHAM, AL, 35203-2104, US

(21) Appl. No.: 12/516,756

(22) PCT Filed: Nov. 28, 2007
(86) PCT No.: PCT/GB07/04561

§ 371 Date: May 28, 2009

Related U.S. Application Data
(60) Provisional application No. 60/861,436, filed on Nov. 29, 2006.

(30) Foreign Application Priority Data
Nov. 29, 2006 (GB) 0623892.7

(51) Int. Cl. A61K 31/155; G01N 33/53; C12N 9/99; A61P 3/10

(52) U.S. Cl. 514/635; 435/7.1; 435/184

(57) ABSTRACT

The invention relates to a method for identifying a candidate agent for use in a medicament for diabetes or obesity said method comprising (i) providing a candidate inhibitor of PPM phosphatase, (ii) providing a first and a second sample comprising PPM phosphatase, (iii) contacting said candidate inhibitor with said first sample comprising PPM phosphatase, and (iv) assaying said first and second samples for PPM phosphatase activity, wherein said PPM phosphatase is selected from the group consisting of PPM1E, PPM1F, PPM1J, PPM1K, PPM1L and PPM1M, wherein if the PPM phosphatase activity is lower in said first sample than in said second sample then said candidate inhibitor is identified as a candidate agent for use in a medicament for diabetes or obesity, preferably type II diabetes. The invention also relates to the use of metformin and phenformin as inhibitors of PPM phosphatases.

### FIELD OF THE INVENTION

The invention is in the field of phosphatase action and inhibition, and also relates to disorders of glucose metabolism/regulation such as diabetes and obesity.

### BACKGROUND TO THE INVENTION

Metformin is a biguanide compound known for the treatment of diabetes. The target of metformin is not known. Metformin can act as a mitochondrial toxin.

Phenformin is an analogue of metformin and is also a biguanide compound. Phenformin has been used in treatment of diabetes. The target of phenformin is not known. Phenformin is a potent mitochondrial toxin. Phenformin exhibits side effects which include severe lactic acidosis. This can be, and has been, fatal. The presence of these side effects such as lactic acidosis have led to the withdrawal of phenformin as a diabetes therapeutic in a majority of territories worldwide.

The AMP-activated protein kinase has long been regarded as one of the key regulators of cellular energy, and has been shown to be activated by at least two upstream kinases, LKB1 and CaMKKβ. The protein phosphatase responsible for dephosphorylation of AMPK was thought to be a member of the PPM family protein phosphatases, by virtue of the fact that bacterially expressed PP2Cα was able to decrease the phosphorylation state of AMPK, an effect which was inhibited by AMP, as well as data on okadaic acid insensitivity.

Many members of the PPM family of protein phosphatases have been identified but the identity of the PPM family member(s) responsible for AMPK dephosphorylation remains unknown in the art. Many studies have indicated that the anti-diabetic drug metformin activates AMPK by increasing phosphorylation of its catalytic T-loop residue, Thr172. Metformin, through activation of AMPK, decreases hepatic glucose production and increases glucose utilisation, but the mechanism by which metformin activates AMPK is unknown. Koh et al (2002 Current Biology vol 12 pp 317-321) disclose that p21 activated kinase PAK is negatively regulated by POPX1 and POPX2, a pair of serine/threonine phosphatases of the PP2C family.

WO2006/091701 discloses methods and compositions for modulating cell death with survival or death kinases or phosphatases. This document presents very large lists of alternative kinases and phosphatases which might be able to modulate cell death or survival if they had some role in control of apoptosis.

The present invention seeks to overcome problem(s) associated with the prior art.

### SUMMARY OF THE INVENTION

The present inventors have discovered a surprising effect of biguanide compounds on phosphatase activity. Specifically, it has been shown that the biguanide compounds commonly used in the treatment of diabetes (such as type II diabetes) and obesity are in fact inhibitors of protein phosphatase activity.

In particular, the inventors have specifically defined the class of phosphatases which are affected as PPM type phosphatases, and have identified within this family of enzymes which of their activities are inhibited.

Thus, the inventors disclose for the first time that protein phosphatases are a point of therapeutic intervention, particularly certain PPM phosphatases.

The invention is based on these surprising findings.

Thus in a broad aspect the invention relates to the use of biguanide compounds as inhibitors of protein phosphatase activity.

In another broad aspect the invention relates to the use of phosphatase inhibitor(s), in particular PPM phosphatase inhibitor(s), in the treatment or prevention of disorders of glucose regulation such as diabetes and/or obesity.

In one aspect the invention relates to a method for identifying a candidate agent for use in a medicament for diabetes or obesity said method comprising

(i) providing a candidate inhibitor of PPM phosphatase,
(ii) providing a first and a second sample comprising PPM phosphatase,
(iii) contacting said candidate inhibitor with said first sample comprising PPM phosphatase, and
(iv) assaying said first and second samples for PPM phosphatase activity, wherein if the PPM phosphatase activity is lower in said first sample than in said second sample then said candidate inhibitor is identified as a candidate agent for use in a medicament for diabetes or obesity.

Preferably the phosphatase is a PPM BIGi (biguanide inhibited) family member. Preferably the phosphatase is encoded by the PPM1E, PPM1F, PPM1J, PPM1K, PPM1L, PHLPP or PHLPP2 genes. Preferably the phosphatase is encoded by the PPM1E, PPM1F, PPM1J, PPM1K, or PPM1L genes. Preferably the PPM phosphatase is PPM1E and/or PPM1F; preferably the PPM phosphatase is PPM1E.

As used herein, the term “agent” or “candidate inhibitor” may be a single entity or it may be a combination of entities. The agent may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule—which may be a sense or an anti-sense molecule. The agent may even be an antibody. The agent may be designed or obtained from a library of compounds, which may comprise peptides, as well as other compounds, such as small organic molecules.

Assaying PPM phosphatase activity is described in detail herein, and examples of suitable assay formats are provided in particular in Example 2.

The sample may be any suitable sample comprising PPM phosphatase. This may be a sample of recombinant enzyme or may be a sample of purified enzyme or may be a simple extract or lysate which comprises PPM phosphatase. Clearly it is important that the sample comprises active PPM protein phosphatase/PPM protein phosphatase activity, otherwise it would not be possible to distinguish an agent having an inhibitory effect from one with no inhibitory effect. This can be easily verified using the assays such as phosphor-casein assays as described herein.

Preferably the disorder is diabetes, more preferably type II diabetes.

Preferably said candidate inhibitor is a biguanide; preferably said candidate inhibitor is a metformin or phenformin analogue or derivative.

In another aspect, the invention provides use of metformin or phenformin in the inhibition of PPM type protein phosphatase.

Preferably the PPM type protein phosphatase has one or more of the characteristics of PPM phosphatases set out herein, preferably two or more, preferably three or more, preferably four or more, preferably all of the characteristics of PPM phosphatases set out herein. Preferably said PPM type protein phosphatase is PPM1E or PPM1F.

In another aspect, the invention provides metformin or phenformin for use in inhibition of PPM phosphatase.

In another aspect, the invention provides use of metformin or phenformin in a composition for use as a PPM phosphatase inhibitor. Furthermore, the invention provides use of metformin or phenformin in manufacture of a composition for use as a PPM phosphatase inhibitor.

In another aspect, the invention provides use of phenformin or an analogue thereof in the enhancement or maintenance of phosphorylation of AMPK.

In another aspect, the invention provides use of PPM1E or PPM1F in the dephosphorylation of AMPK.

In another aspect, the invention provides use of metformin or phenformin in the activation of p21-activated kinase (PAK).

In another aspect, the invention provides use of metformin or phenformin in the inhibition of dephosphorylation of Ca2⁺/Calmodulin dependent kinase II (CaMKII).

In another aspect, the invention provides an agent identified by a method as described above for use as a medicament.

In another aspect, the invention provides use of an agent identified by a method as described above for the manufacture of a medicament for diabetes or obesity.

In another aspect, the invention provides an agent identified by a method as described above for use in the treatment of diabetes or obesity.

In another aspect, the invention provides a method of treatment or prevention of diabetes or obesity comprising administering a composition containing a medicament as described above to a subject, wherein said medicament does not comprise metformin or phenformin.

In another aspect, the invention provides a method of treatment or prevention of diabetes or obesity comprising inhibiting PPM phosphatase in a subject. Preferably said PPM phosphatase is selected from the group consisting of PPM1E, PPM1F, PPM1J, PPM1K, PPM1L, PPM1M, PHLPP and PHLPP2. Preferably said PPM phosphatase is selected from the group consisting of PPM1E, PPM1F, PPM1J, PPM1K, PPM1L, or PPM1M. Preferably said PPM phosphatase is PPM1E and/or PPM1F. Preferably such inhibition is not by metformin or phenformin.

### DETAILED DESCRIPTION OF THE INVENTION

Metformin is one of the main drugs used in the treatment of type 2 diabetes and increases the activity of AMPK, although its mechanism of action is unclear. AMPK is usually activated in response to increases in the levels of AMP and by phosphorylation at a threonine residue within the catalytic site of the α subunit: Examination of protein phosphatase activities after incubation of HEK293 and HeLa cells with the metformin analogue phenformin revealed that the magnesium ion-dependent, okadaic acid resistant casein phosphatase activity was decreased by ˜20% in response to phenformin or metformin. Further investigation showed that the activity of two closely related PPM family protein phosphatases, PPM1E and PPM1F were inhibited following incubation of HEK293 cells with phenformin, revealing that they may be targeted directly and/or indirectly by phenformin and lead to an increase in AMPK activity. The present invention is based on these surprising findings.

The term ‘agent’ or ‘candidate inhibitor’ has its normal meaning in the art and may refer to any chemical entity such as an organic or inorganic compound, or a mixture thereof. Preferably the agent may be an small chemical entity. Preferably the substance may be a macromolecule such as a biological macromolecule e.g. a nucleic acid or polypeptide. By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof).

Typically, the agent will be an organic compound. Preferred agents are water soluble. Preferably agents of the invention are metformin analogues or phenformin analogues.

Preferably the agent(s) of the invention comprise means for transport into the cell, and may comprise means for transport into the mitochondria. More preferably the agent(s) of the invention are excluded from mitochondria.

Metformin/Phenformin/Analogues

Preferably PPM inhibitors according to the present invention are biguanide compounds. Examples include metformin, phenformin or buformin.

Preferably PPM inhibitors according to the present invention comprise metformin or an analogue thereof, or phenformin or an analogue thereof.

Analogues of metformin include phenformin, which is a preferred compound of the present invention due to its PPM phosphatase inhibitory activity.

Phenformin is phenylethylbiguanide. Phenformin is a biguanide hypoglycemic agent with properties similar to those of metformin. It must be noted that in many jurisdictions phenformin is considered to be associated with an unacceptably high incidence of lactic acidosis, which is often fatal. Thus, preferably phenformin is not administered to human or animal subjects. Thus, preferably the PPM phosphatase inhibitor is not phenformin for medical applications of the invention.

Metformin (C₄H₁₁N₅) is 1-(diaminomethylidene)-3,3-dimethyl-guanidine. Metformin is an anti-diabetic drug from the biguanide class. Metformin is widely available under trade names such as Glucophage, Diabex, Diaformin, Fortamet, Riomet, Glumetza and others. Metformin is a preferred compound of the invention due to its PPM phosphatase inhibitory activity and due to its lower toxicity.

### Chemical Derivatives

The invention also relates to derivatives of the compounds, in particular derivatives of metformin and/or phenformin. The term “derivative” as used herein includes chemical modification of an agent. Illustrative of such chemical modifications would be replacement of hydrogen by a halo group, an alkyl group, an acyl group or an amino group.

Salts/Esters

The compounds of the invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

In all aspects the invention includes, where appropriate, all enantiomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art.

Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Solvates

The present invention also includes the use of solvate forms of the compounds of the present invention. The terms used in the claims encompass these forms.

### Polymorphs

The invention furthermore relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

The invention further includes the compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### PPM Family of Protein Phosphatases

The PPM family of protein phosphatases comprise a group of serine/threonine phosphatases, many of which are dependent on Mg2+ or Mn2+ for their activity. Although there is no sequence identity between PPP and PPM family protein phosphatases, they have remarkably similar three-dimensional structures and catalytic mechanisms. The PPM family of protein phosphatases comprises at least 16 members (16 genes encoding PPMs; some alternatively spliced to produce different protein variants (eg B1 and B2)). Although these protein phosphatases are rather divergent in structure, they operate with a very similar catalytic mechanism. According to convention, the proteins and gene names may differ for example as set out below (e.g. the PPM1E gene encodes POPX1 protein). However, if a protein is referred to by the gene name then this will be understood to refer to the phosphatase encoded by said gene (e.g. ‘PPM1E phosphatase’ refers to the phosphatase encoded by the PPM1E gene i.e. POPX1 protein).

### PPM1A

PPM1A, also called PP2Cα, consists of 2 isoforms, PP2Cα1 (PPM1A1) and PP2Cα2 (PPM1A2), which have molecular masses of 42 and 36 kDa respectively and both of which exist as monomers. PP2Cα was first expressed in E-coli and it was found that, in vitro, recombinant PP2Cα was able to dephosphorylate the AMP-activated protein kinase (AMPK). This dephosphorylation event was not sensitive to the toxin okadaic acid and had a requirement for Mg²⁺. In addition, PP2Cα has been shown to be a positive regulator of insulin sensitivity through direct activation of PI 3-kinase activity in adipocytes and has been suggested as a negative regulator of stress response pathways through dephosphorylation and inactivation of mitogen-activated protein kinase kinases (MAPKKs), as well as p38 MAPK. When cells were stimulated with stress, a direct interaction between p38 and PP2Cα could be detected.

### PPM1B

PPM1B, also called PP2Cβ, consists of at least 2 isoforms, PP2Cβ1 (PPM1B1) and PP2Cβ2 (PPM1B2), with molecular masses of 43 and 53 kDa respectively and like PP2Cα, these enzymes exist as monomers. PP2Cβ has been shown to be a negative regulator of the stress response pathway through dephosphorylation of p38, and has also been implicated in the dephosphorylation of TAK1, an enzyme involved in activation of the INK and MAPK pathways. PP2Cβ has been shown to play a role in the dephosphorylation of cyclin-dependent phosphatases.

### PPM1G

PPM1G, also called PP2Cγ or FIN13 is a monomeric protein phosphatase containing a fused collagen homology domain of molecular mass 59 kDa which has been shown to be able to negatively regulate cell proliferation by causing cell cycle arrest in G₁/S phase. In adult tissues, PPM1G is expressed mainly in the testis, but is highly expressed in a number of tissues undergoing proliferation. These include the developing embryo, the uterus at pregnancy, the placenta and in the ovaries of sexually immature mice after stimulation of folliculogenesis with diethylstilbestrol (DES). PPM1G has a C-terminal nuclear localisation signal, and differs from other members of the PPM family in that it possesses a large internal acidic domain, which is thought to be involved in conferring substrate specificity, since PP2Cγ appears to have a preference for mainly basic proteins. PPM1G has been shown to be inhibited by calcium but this is unlikely to be a mode of regulation owing to the high micromolar concentrations required for inhibition. PPM1G has been implicated in the assembly of the spliceosome and has been shown to interact with components of the pre-mRNA splicing factor.

ILKAP

ILKAP (for Integrin-linked kinase 1-associated phosphatase), also called PP2Cδ, is a monomeric protein of molecular mass 43 kDa and was identified in a yeast two-hybrid screen baited with integrin-linked kinase 1 (ILK1). ILKAP, but not a catalytically inactive mutant of ILKAP, strongly inhibited insulin-like growth factor 1-stimulated GSK3β phosphorylation on Ser9, but did not affect phosphorylation of PKB at Ser473, suggesting that ILKAP selectively affects ILK-mediated GSK3β signalling. In addition, anchorage-independent growth of prostate carcinoma LNCaP cells was inhibited by ILKAP, suggesting a critical role for ILKAP in the suppression of cellular transformation, and that ILKAP plays an important role in inhibiting oncogenic transformation.

### PPM1D

PPM1D, also called Wip1 (for wildtype p53-induced phosphatase 1), is a monomeric protein with a molecular mass of 66 kDa and was first identified in a screen for p53 target genes. Its expression was shown to be rapidly induced by ionising radiation in a p53-dependent manner and it was suggested that p53 might mediate part of its cell cycle inhibitory activities through the induction of PPM1D. The Wip1 promoter region does not contain any of the traditional p53-response elements, but does instead contain potential binding sites for transcription factors that include NF-κB, E2F, c-Jun and members of the ATF/CREB family. Like other PP2C family members, PPM1D is able to dephosphorylate members of the p38 MAPK family. PPM1D has a role in down-regulating p38-p53 signalling during the recovery phase of cells damaged by UV-irradiation. In addition, PPM1D is also induced by other environmental stresses, such as anisomycin, hydrogen peroxide, and methyl methane sulfonate. For the UV-induction of PPM1D, p38 activity is required as well as p53, and PPM1D inactivates p38 by dephosphorylation at its conserved threonine residue, whilst decreasing UV-induced p53 phosphorylation at those residues reported to be phosphorylated by p38. PPM1D expression also suppresses both p53-mediated transcription and apoptosis in response to UV-radiation.

PPM1E and PPM1F

PPM1E, also called POPX1, is a monomeric protein of 83 kDa containing a fused PAK-interacting guanine nucleotide exchange factor (PIX) binding domain, and has been suggested to be involved in the negative regulation of PAK. PPM1E was identified as a PIX-interacting protein in a two-hybrid screen using PIX as bait. PPM1F, also called POPX2, was identified in the same screen and the two exhibit 66% similarity over the core phosphatase domain and homologous flanking sequence. Both PPM1E and PPM1F have been shown to dephosphorylate and inactivate the p21 (cdc42/Rac)-activated kinase (PAK), as well as having the ability to inhibit actin stress fibre breakdown and inhibit morphological changes driven by active cdc42. PPM1F is also known as CaMKIIPase, or hFEM2, and has been shown to be the major phosphatase responsible for dephosphorylation of the Ca²⁺/Calmodulin dependent protein kinase II at its autophosphorylation site, Thr286. It has been shown that PPM1F interacts directly with CaMKII in vitro and it is suggested that PPM1F plays a key role in its regulation.

### PDPC1 and PDPC2

PDPC1 (Pyruvate Dehydrogenase Phosphatase Complex 1) and PDPC2 are heterodimeric proteins with molecular masses of 61 and 60 kDa respectively and are two of the few mammalian phosphatases which reside within the mitochondrial matrix space. PDPC1 has been shown to be activated in response to Ca²⁺, whilst PDPC2 has been shown to be Ca²⁺-insensitive, but to be sensitive to the biological polyamine, spermine, which has no effect on PDPC1. The pyruvate dehydrogenase complex is a large multi-enzyme complex that is composed of three catalytic components: pyruvate dehydrogenase (E1), dihydrolipoamide transacetylase (E2), and dihydrolipoamide dehydrogenase (E3). The complex is built around a core of 60 E2 subunits to which 30 subunits of E1 and 6-12 E3 subunits are bound. The complex is inactivated by phosphorylation on three serine residues in the E1 component and is reactivated by dephosphorylation by the PDPC isoforms. In the mitochondria, both the kinase and the phosphatase are constitutively active and this determines the proportion of the PDC in the inactive state. Clearly, regulation of the activities of PDPC1 and PDPC2 must be very tightly controlled and recent studies suggest that starvation and diabetes decrease the levels of PDP in heart and kidney. Interestingly, treatment with insulin was shown to increase the levels of PDPC2, suggestive of the fact that insulin might play a role in the long term regulation of the pyruvate dehydrogenase complex.

### PHLPP

PHLPP (for PH-domain leucine-rich protein phosphatase) is a novel phosphatase of around 140 kDa which was identified in a screen of the human genome for a protein phosphatase linked to a PH domain, and which dephosphorylates Thr473 on PKB. Consistent with its role in dephosphorylating PKB, a number of colon cancer and glioblastoma cell lines have decreased levels of PHLPP and reintroduction of PHLPP into these cell lines decreases their growth rate. PHLPP therefore has a role in promoting apoptosis and suppressing tumour growth. A second isoform of PHLPP is encoded in the human genome. PHLPP and PHLPP2 are of less interest due to their association with PKB; thus, suitably the phosphatase of the invention is not PHLPP or PHLPP2.

### PPM1K

PPM1K is a PPM serine/threonine protein phosphatase family member which has recently been identified and placed in the NCBI and EBI databases under ID numbers NP 689755, ENSP00000295908, ENSP00000324761, Q56AN8, Q8IUZ7, Q49AB5.

In summary, PPMs not inhibited by phenformin/metformin include PPM1A, PPM1B (Bland B2), PPM1G, 1LKAP, PPM1D, NERPP-2C, PDPC1 and PDPC2.

Preferably the PPM is a PPM BIGi (biguanide inhibited) family member. BIGi family members include phosphatases encoded by the PPM1E, PPM1F, PPM1J, PPM1K, PPM1L, PHLPP and PHLPP2 genes, and any other phosphatase inhibited by biguanide such as metformin and/or phenformin assayed as disclosed herein. Preferably the PPM phosphatase is selected from the group consisting of PPM1E, PPM1F, PPM1J, PPM1K, PPM1L, PPM1M, PHLPP and PHLPP2, preferably the PPM phosphatase is selected from the group consisting of PPM1E, PPM1F, PPM1J, PPM1K, PPM1L, and PPM1M, or is a combination of one or more phosphatases selected therefrom; preferably the PPM phosphatase is PPM1E or PPM1F; preferably the PPM phosphatase is PPM1E.

### Summary of PPM: Family Members:

| | | Genbank Protein Acc. no., |
|---|---|---|
| | | (mRNA accession no.) |
| Gene name | Protein name(s) | Ensembl Peptide ID., (Gene ID) |
| PPM1E | POPX1 | NP_055721, (NM_014906) |
| | PP2CH | ENSP00000312411, (ENSG00000175175) |
| | | Q8WY54, Q8WY54_2, Q8WY54_1, |
| | | Q8WY54_3 |
| PPM1F | POPX2 | NP_055449, P49593, (NM_014634) |
| | CaM-KPase | ENSP00000263212, (ENSG00000100034) |
| | hFEM2 | P49593, Q0VGL7, Q6IPCO |
| PPM1J | PP2Czeta | NP_005158, (NM_027982) |
| | | ENSP00000308926; ENSP00000353088, |
| | | (ENSG00000155367) 2 isoforms |
| | | Q6DKJ7, Q5JR12 |
| PPM1K | | NP_689755, (NM 152452) |
| | | ENSP00000295908, ENSP00000324761 |
| | | (ENSG00000163644) 2 isoforms |
| | | Q56AN8, Q8IUZ7, Q49AB5 |
| PPM1L | PP2Cepsilon | NP_640338, (NM_178726) |
| | PP2CL | ENSP00000295839, (ENSG00000163590) |
| | | Q5SGD2, Q2M3J2 |
| PPM1M | PP2Ceta | Q96MI6 |
| PHLPP | PHLPPalpha | NP_919431, (NM_194449) |
| | SCOP | ENSP00000262719 (ENSG00000081913) |
| | PLEKHE1 | O60346 |
| PHLPP2 | PHLPPbeta | NP_055835, (NM_015020) |

### Preferred Characteristics of PPM Phosphatases are Presented:

Preferably the PPM phosphatase is a magnesium (Mg2+) or manganese (Mn2+) dependent phosphatase, preferably manganese dependent.

Preferably the PPM phosphatase is okadaic acid resistant.

Preferably the PPM phosphatase has casein (e.g. phospho-casein) phosphatase activity. Preferably the PPM phosphatase comprises a PIX-binding domain.

Preferably the PPM phosphatase is PPM1F or PPM1E or a mixture thereof.

Preferably the PPM phosphatase is PPM1E.

Preferably the PPM phosphatase has an amino acid sequence selected from:

PPM1E. NP_055721
MAGCIPEEKTYRRFLELFLGEFRGPCGGGEPEPEPEPEPEPEPEPESEPE
PEPELVEAEAAEASVEEPGEEAATVAATEEGDQEQDPEPEEEAAVEGEEE
EEGAATAAAAPGHSAVPPPPPQLPPLPPLPRPLSERITPRPLSERITREE
VEGESLDLCLQQLYKYNCPSFLAAALARATSDEVLQSDLSAHYIPKETDG
TEGTVEIETVKLARSVFSKLHEICCSWVKDFPLRRRPQLYYETSIHAIKN
MRRKMEDKHVCIPDFNMLFNLEDQEEQAYFAVFDGHGGVDAAIYASIHLH
VNLVRQEMFPHDPAEALCRAFRVTDERFVQKAARESLRCGTTGVVTFIRG
NMLHVAWVGDSQVMLVRKGQAVELMKPHKPDREDEKQRIEALGGCIVWFG
AWRVNGSLSVSRAIGDAEHKPYICGDADSASTVLDGTEDYLILACDGFYD
TVNPDEAVKVVSDHLKENNGDSSMVAHKLVASARDAGSSDNITVIVVFLR
DMNKAVNVSEESDWTENSFQGGQEDGGDDKENHGECKRPWPQHQCSAPAD
LGYDGRVDSFTDRTSLSPGSQINVLEDPGYLDLTQIEASKPHSAQFLLPV
EMFGPGAPKKANLINELMMEKKSVQSSLPEWSGAGEFPTAFNLGSTGEQI
YRMQSLSPVCSGLENEQFKSPGNRVSRLSHLRHHYSKKWHRFRFNPKFYS
FLSAQEPSHKIGTSLSSLTGSGKRNRIRSSLPWRQNSWKGYSENMRKLRK
THDIPCPDLPWSYKIE
PPM1E. ENSP00000312411
MAGCIPEEKTYRRFLELFLGEFRGPCGGGEPEPEPEPEPEPEPESEPE
PEPELVEAEAAEASVEEPGEEAATVAATEEGDQEQDPEPEEEAAVEGEEE
EEGAATAAAAPGHSAVPPPPPQLPPLPPLPRPLSERITREEVEGESLDLC
LQQLYKYNCPSFLAAALARATSDEVLQSDLSAHYIPKETDGTEGTVEIET
VKLARSVFSKLHEICCSWVKDFPLRRRPQLYYETSIHAIKNMRRKMEDKH
VCIPDFNMLFNLEDQEEQAYFAVFDGHGGVDAAIYASIHLHVNLVRQEMF
PHDPAEALCRAFRVTDERFVQKAARESLRCGTTGVVTFIRGNMLHVAWVG
DSQVMLVRKGQAVELMKPHKPDREDEKQRIEALGGCVVWFGAWRVNGSLS
VSRAIGDAEHKPYICGDADSASTVLDGTEDYLILACDGFYDTVNPDEAVK
VVSDHLKENNGDSSMVAHKLVASARDAGSSDNITVIVVFLRDMNKAVNVS
EESDWTENSFQGGQEDGGDDKENHGECKRPWPQHQCSAPADLGYDGRVDS
FTDRTSLSPGSQINVLEDPGYLDLTQIEASKPHSAQFLLPVEMFGPGAPK
KANLINELMMEKKSVQSSLPEWSGAGEFPTAFNLGSTGEQIYRMQSLSPV
CSGLENEQFKSPGNRVSRLSHLRHHYSKKWHRFRFNPKFYSFLSAQEPSH
KIGTSLSSLTGSGKRNRIRSSLPWRQNSWKGYSENMRKLRKTHDIPCPDL
PWSYKIE

These two PPM1E sequences NP_{—}055721 and ENSP00000312411 are 98% identical.

Alternatively the PPM1E sequence may be selected from Q8WY54_{—}2, Q8WY54_{—}1, or Q8WY54_{—}3. Q8WY54_{—}2 has an additional EP compared to the most suitable sequences listed above (MAGCIPEEKTYRRFLELFLGEFRGPCGGGEP . . . ), whereas Q8WY54_{—}1 and Q8WY54_{—}3 possess other variations in the amino terminal region.

PPM1F. NP_055449 and ENSP00000263212
MSSGAPQKSSPMASGAEETPGFLDTLLQDFPALLNPEDPLPWKAPGTVLS
QEEVEGELAELAMGFLGSRKAPPPLAAALAHEAVSQLLQTDLSEFRKLPR
EEEEEEEDDDEEEKAPVTLLDAQSLAQSFFNRLWEVAGQWQKQVPLAARA
SQRQWLVSIHAIRNTRRKMEDRHVSLPSFNQLFGLSDPVNRAYFAVFDGH
GGVDAARYAAVHVHTNAARQPELPTDPEGALREAFRRTDQMFLRKAKRER
LQSGTTGVCALIAGATLHVAWLGDSQVILVQQGQVVKLMEPHRPERQDEK
ARIEALGGFVSHMDCWRVNGTLAVSRAIGDVFQKPYVSGEADAASRALTG
SEDYLLLACDGFFDVVPHQEVVGLVQSHLTRQQGSGLRVAEELVAAARER
GSHDNITVMVVFLRDPQELLEGGNQGEGDPQAEGRRQDLPSSLPEPETQA
PPRS
PPM1F. Q6IPC0
MSSGAPQKSSPMASGAEETPGFLDTLLQDFPALLNPEDPLPWKAPGTVLS
QEEVEGELAELAMGFLGSRKAPPPLAAALAHEAVSQLLQTDLSEFRKLPR
EEEEEEEDDDEEKAPVTILLDAQSLAQSFFNRLWEVAGQWQKQVPLAARA
SQRQWLVSIHAIRNTRRKMEDRHVSLPSFNQLFGLSDPVNRAYFAVFDGH
GGVDAARYAAVHVHTNAARQPELPTDPEGALREAFRRTDQMFLRKAKRER
LQSGTTGVCALIAGATLHVAWLGDSQVILVQQGQVVKLMEPHRPERQDEK
ARIEALGGFVSHMDCWRVNGTLAVSRAIGDVFQKPYVSGEADAASRALTG
SEDYLLLACDGFFDVVPHQEVVGLVQSHLTRQQGSGLRVAEELVAAARER
GSHDNITVMVVFLRDPQELLEGGNQGEGDPQAEGRRQDLPSSLPEPETQA
PPRS
PPM1F. Q0VGL7
MSSGAPQKSSPMASGAEETPGFLDTLLQDFPALLNPEDPLPWKAPGTVLS
QEEVEGELAELAMGFLGSRKAPPPLAAALAHEAVSQLLQTDLSEFRKLPR
EEEEEEEDDDEEEKAPVTLLDAQSLAQSFFNRLWEVAGQWQKQVPLAARA
SQRQWLVSIHAIRNTRRKMEDRHVSLPSFNQLFGLSDPVNRAYFAVFDGH
GGVDAARYAAVHVHTNAARQPELPTDPEGALREAFRRTDQMFLRKAKRER
LQSGTTGVCALIAGATLHVAWLGDSQVILVQQGQVVKLMEPHRPERQDEK

Preferably the phosphatase protein has a sequence which is at least 80% identical to one of these sequences; preferably at least 85% identical; preferably at least 90% identical; preferably at least 95% identical; preferably at least 96% identical; preferably at least 97% identical; preferably at least 98% identical; preferably at least 99% identical to one of these sequences, provided in each case that the phosphatase protein has retained phosphatase activity. This may be easily verified using the assays described herein.

### Inhibitors and Assay of PPM Phosphatase Activity

References to phosphatase inhibitors, in particular PPM phosphatase inhibitors, should be interpreted in accordance with the teachings regarding phosphatases i.e. preferably the phosphatase inhibitor(s) are inhibitors of magnesium or manganese dependent PPM phosphatase, preferably inhibitors of okadaic acid resistant PPM phosphatase, preferably inhibitors of PPM phosphatase casein phosphatase activity, preferably inhibitors of PPM phosphatase comprising a PIX-binding domain, preferably inhibitors of PPM1E or PPM1F phosphatase or a mixture thereof, preferably inhibitors of PPM1F phosphatase, preferably inhibitors of PPM1E phosphatase.

Preferably the phosphatase inhibitor of the invention is an inhibitor of PPM phosphatase and preferably has no significant effect on PP2A phosphatase activity, preferably no detectable effect on PP2A phosphatase activity, preferably no detectable effect on PP2A phosphatase activity when assayed as described herein using phosphorylase a as substrate, (in particular when PP1 is inhibited by use of I-2 inhibitor—see Example 2).

Preferably the PPM inhibitor has no significant effect on PP1 activity, preferably no detectable effect on PP1 activity, preferably no detectable effect on PP1 activity when assayed as described in Example 2.

Preferably the PPM inhibitor has no significant effect on PP5 activity, preferably no detectable effect on PP5 activity, preferably no detectable effect on PP5 activity when assayed as described in Example 2.

Inhibiting PPM phosphatase is preferably accomplished by administration of a PPM inhibitor. ‘Inhibition’ may also comprise reduction or elimination of PPM activity or interference/intervention with regard to levels of PPM phosphatase. For example, suppression or inhibition of expression of PPM phosphatase, suppression or inhibition of transcription and/or translation of PPM phosphatase or downregulation of PPM phosphatase itself (whether by modulating the enzyme such as preventing its activation or causing its inactivation, or by accelerating its degradation or other such technique).

Thus, ‘inhibition’ refers to the lowering, quashing, removal, or other such mode of suppressing or reducing PPM phosphatase activity. Inhibitor is preferably an inhibitor identified according to an assay disclosed herein. Other modes of inhibition of PPM phosphatase may be employed. These may involve manipulation of the activator(s) or regulator(s) of PPM. Alternatively these may involve PPM knock-downs such as siRNA knock-down of PPM activity. Preferably siRNAs used to inhibit PPM activity are PPM1E or PPM1F siRNAs.

Assay of PPM phosphatase activity may be by any suitable assay. Numerous possible formats are described in detail in the examples section.

Preferred features of the assay are that it comprises Mg2⁺ ions or Mn2⁺ ions, preferably Mg2⁺ ions; preferably MgAc (magnesium acetate); preferably 10 mM MgAc. This has the benefit of being permissive for activity since PPM phosphatases are Mg/Mn dependent. Suitably the assay comprises Mn2⁺ ions; suitably MnCl₂ (manganese chloride); suitably 2 mM manganese (II) chloride. Suitably assays comprise both Mg2⁺ ions and Mn2⁺ ions.

Preferably the assay comprises okadaic acid, preferably 5 μM okadaic acid. This has the benefit of inhibiting PP2A.

Preferably the assay comprises one or more inhibitor(s) of other phosphatases which may act on the particular substrate being used so as not to confound the results i.e. to try to ensure that the assay accurately reads out PPM phosphatase activity/inhibition rather than the activity/inhibition of another phosphatase such as a non-PPM phosphatase. Such inhibitors and the phosphatases which they inhibit are known and exemplary inhibitors are described herein, in particular in the examples section, together with an indication of which enzyme(s) they inhibit.

Preferably the assay is conducted using casein as a substrate (phospho-casein). Preferably this casein is labelled with ³²P to facilitate detection of phosphate removed by PPM action.

Preferably assay is on FPLC purified phosphatase.

Preferably assay is on PPM phosphatase such as PPM1E/PPM1F phosphatase expressed in mammalian cells, bacterial cells or other heterologous expression systems provided such material has activity (which is easily tested as set out herein).

Preferably assay is on immunopurified phosphatase. Preferably the phosphatase is immunopurified using anti-PPM1E and/or anti-PPM1F antibody or antibody fragment(s).

Preferably the PPM phosphatase activity of the assay is PPM1E and/or PPM1F.

### General Protein Phosphatase Assay

Protein phosphatases are assayed at 30° C. in a volume of 30 μl, with 1 μM-10 μM 32P-labelled substrate. The 32P-labelled substrate and phosphatase inhibitors/activators are diluted separately in buffer C. The protein phosphatase is diluted into buffer B. The assay is performed by mixing 10 μl of the diluted phosphatase (or immunopellet in 10 μl buffer B) with 10 μl of the inhibitor/activator or buffer C and incubating the mixture at 30° C. for 10 min. The assay is started by the addition of 10 μl of 32P-substrate. The assay is then incubated for a further time (5-30 min) at 30° C. and stopped by the addition of 100 μl 20% (w/v) trichloroacetic acid. The mixture is vortexed briefly and centrifuged at 14,000×g for 5 minutes. 100 μl of supernatant is recovered and the 32P released is measured by Cerenkov counting on a Wallac 1409 liquid scintillation counter. For assays of immunopelleted phosphatase activity the procedure is exactly as described above except that the washed immune pellet is resuspended in 10 μl buffer B and the assay was incubated with shaking at 1200 rpm at 30° C.

| | |
|---|---|
| Buffer A | 50 mM Tris-HCl pH 7.5, 0.1 mM EGTA, 0.1% (v/v) 2- |
| | mercaptoethanol. |
| Buffer B | Buffer A containing 1 mg/ml BSA. |
| Buffer C | Buffer A containing 0.01% (v/v) Brij-35. |

32P-labelled casein substrate is partially hydrolysed bovine milk casein (Sigma, Poole UK) labelled with [γ32P]ATP using the catalytic subunit of protein kinase A.

Other 32P-labelled substrates may be used provided the phosphatase of interest (e.g. PPM1E and PPM1F) acts to dephosphorylate them.

### In Vitro PPM Assays

PPM phosphatase activity may be determined by the release of [32P]-orthophosphate from a glutathionine-5-transferase-peptide substrate GST-(GGGGRRAT[p]VA)3 substrate in the presence of okadaic acid to inhibit PP1 and PP2A like activities.

The phosphatase may be provided by immunopelleting as in example 10 or more conveniently provided by expression of a PPM1E and purification of the expressed protein by standard techniques (such as using purification tag(s) such as 6his or GST fused to the PPM1E polypeptide) such as in example 12. In case any guidance is needed the amino acid sequence(s) of preferred PPM1E variants are provided in the text.

GST-(GGGGRRAT[p]VA)₃ phosphatase substrate is suitably prepared by phosphorylation with protein kinase A (PKA). 2 mg of bacterially expressed GST-(GGGGRRATVA)₃ is incubated with I-2 mU PKA overnight at 30° C. with gentle shaking in a buffer consisting of 50 mM Tris-HCl pH 7.0, 0.1 mM EGTA, 10% glycerol, 10 mM magnesium acetate, 0.1% (v/v) 2-mercaptoethanol, 0.1 mM [gamma32P] ATP.

GST-(GGGGRRATVA)3 protein sequence (PreScission
Protease site underlined):
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLEVLFQGPLGSGGGGRRATVAGGGGRRATV
AGGGGRRATVAGGG

Labelled GST-(GGGGRRAT[p]VA)3 was separated from unincorporated radionucleotide by column chromatography on a glutathione-Sepharose column, eluting in 50 mM Tris-HCl pH 7.0, 0.1 mM EGTA, 10% glycerol, 10 mM magnesium acetate, 0.1% (v/v) 2-mercaptoethanol, 20 mM glutathione. GST-(GGGGRRAT[p]VA)3 was diluted to 1-4 μM prior to addition during assays.

Phosphatases are assayed in a total volume of 30 μl at 30° C. for ten minutes, with constant shaking. Assays contained the phosphatase diluted in 20 μl buffer, which was incubated at 30° C. for two minutes prior to addition of 10 μl 32P-labelled GST-(GGGGRRAT[p]VA)₃ phosphatase substrate. After ten minutes, reactions were terminated by addition of 100 μl 20% trichloracetic acid. Tubes were then vortexed for a further minute to ensure complete mixing and centrifuged at 16,000×g for 5 minutes at room temperature. 100 μl supernatant was removed from each reaction into a new Eppendorf tube and counted by Cerenkov counting in a liquid scintillation counter.

Acid-molybdate extractions reveal very little contaminating protease activity.

Phosphatase assay composition (final concentrations):

### 50 mM Tris-HCl pH 7.0

### 0.1 mM EGTA

10 mM magnesium acetate
2 mM manganese (II) chloride
5 μM okadaic acid
0.1% (v/v) 2-mercaptoethanol

Mass of phosphatase used in the assays is typically 10 ng-10 μg depending on the preparation.

### Non-Radioactive Assays

The release of phosphate after the 10 minute assay (before the addition of trichloracetic acid) may be measured by the addition of 150 μl of 1 N HCl containing 10 mg/ml ammonium molybdate and 0.38 mg/ml malachite green to a 100 μl assay. After 20 minutes at room temperature (e.g. 18-25° C.), the absorbance is measured at 620 nm. With 250 μl the absorbance may be read on a plate reader. However, the assay may be 10-100 fold less sensitive than radioactive based assays and therefore one may use 10-100 fold more phosphatase in the assay to compensate if necessary, or may simply take into account the lower readout in assessing the results.

### Assay for PPM1E

PPM1E is immunopelleted with antibodies raised the amino acid sequence KTHDIPCPDLPWSY and the phosphatase activity in the immunopellet is measured in a protein phosphatase assay using 32P-labelled casein as substrate in the presence of 10 mM Mg2+ or Mn2+ ions and 5 μM okadaic acid.

Other antibodies that recognise the PPM1E protein(s) encoded by the gene ENSG00000175175 may be used.

### Assay for PPPM1F

PPM1F is immunopelleted with antibodies raised the amino acid sequence LPSSLPEPETQAPPRS and the phosphatase activity in the immunopellet is measured in a protein phosphatase assay using 32P-labelled casein as substrate in the presence of 10 mM Mg2+ or Mn2+ ions and 5 μM okadaic acid.

Other antibodies that recognise the PPM1F protein(s) encoded by the gene ENSG00000100034 may be used.

### AMP-Activated Protein Kinase (AMPK)

AMPK acts as a sensor of cellular energy, switching off ATP-consuming pathways, and switching on catabolic processes which generate ATP. The actions of a number of different metabolic processes are under the control of AMPK, including glucose homeostasis, lipid metabolism, and mitochondrial biogenesis. The enzyme is made up of a heterotrimer, consisting of a catalytic α subunit and regulatory β and γ subunits, each of which are encoded by a number of genes. A number of splice variants of each exist meaning that several combinations of the heterotrimer are possible. The α subunits of AMPK contain the catalytic kinase domain, as well as a domain close to the C-terminus which is both necessary and sufficient for formation of the complex with β and γ subunits. The β subunits contain a carbohydrate binding domain and are thought to be involved in the association, of the complex with glycogen particles. As discussed earlier, one of the physiological targets of AMPK is glycogen synthase, which is also resident at the glycogen particle, and there is a growing body of evidence that high cellular glycogen results in a decrease in AMPK activity. The γ subunits of AMPK contain four repeats of a motif of ˜60 residues termed CBS domains. These motifs act in pairs to each bind one molecule of AMP or ATP in a mutually exclusive manner, consistent with the notion that high concentrations of ATP inhibit activation of AMPK by AMP.

AMPK can be activated in response to exercise and the associated increases in ATP utilisation. During periods of exercise, AMPK inhibits ATP-consuming pathways, whilst activating carbohydrate and fatty acid metabolism in an attempt to restore ATP levels. AMPK can be activated allosterically by AMP as well as by phosphorylation of a threonine residue within the catalytic ‘T loop’ of the α subunit, Thr172. It has been well established that AMPK responds to changes in the cellular ratio of AMP:ATP. The tumour-suppressor kinase LKB1 is the enzyme responsible for phosphorylation of Thr172 in vivo. LKB1 is activated through its interaction with mouse protein 25 (MO25) and STE20-related adaptor protein (STRAD); STRAD is a pseudokinase, whilst MO25 stabilises the interaction between LKB1 and STRAD. In addition, MO25 and STRAD act to localise LKB1 in the cytoplasm. Interestingly, LKB1 is not regulated by stimuli that activate AMPK, nor activated by AMP. A number of researchers are currently investigating the possible modes of regulation of LKB1. In addition to activating AMPK, LKB1 is also able to activate 11 other AMPK-related kinases at their T-loop residues. The Ca²⁺/calmodulin-dependent protein kinase kinase beta (CaMKKβ) also acts as an upstream kinase for AMPK in-vivo, identifying a potential link between muscle contraction and the activation of AMPK. The dephosphorylation of AMPK is thought to be carried out by a PP2C-like enzyme in vivo, although evidence is limited.

AMPK is an indirect target of the anti-diabetic drug metformin, which benefits type 2 diabetics through decreased hepatic glucose production and increased glucose utilisation. Metformin is able to activate AMPK in hepatocytes and consequently decreases the activity of ACC through increased phosphorylation at Ser79, increasing fatty acid oxidation, and suppressing enzymes involved in lipogenesis. Metformin is a member of the biguanide family of drugs, and has been shown to be able to inhibit complex 1 of the respiratory chain.

Numerous downstream targets of AMPK have been identified, including acetyl co-A carboxylase and GLUT4. One of the most important effects is on glycogen synthase (GS), where the use of animal models with no active AMPK proved that this was the primary kinase responsible for the phosphorylation of site 2 on GS. The non-specific activator of AMPK, 5-aminoimidazole-4-carboxamide-1-13-D-ribofuranoside (AICAR) has been shown to decrease GS activity. When examined after AICAR stimulation, GS has increased phosphorylation at site 2 without any effects on sites 3a or 3b in rat muscle. In contrast, after treatment with AICAR, mice have increased glycogen levels, most likely due to the effects of AICAR increasing glucose uptake and a concomitant increase in GS activity by allosteric activation independent-of phosphorylation state. In response to exercise, GS activity is increased, and it is thought that this increase would be to allow glycogen stores to be rapidly repleted following the bout of exercise. However, during very intense exercise, glycogenolysis can reach extremely high rates and so the effect of increased GS activity can be attenuated. It seems likely, therefore, that the effects of AMPK on GS are dependent on the duration and intensity of the exercise, since AMPK is activated during exercise in an intensity-dependent manner. In a mouse model expressing a dominant negative AMPK, GS activity was increased normally in response to 10 minutes of in-vitro contraction, which is highly suggestive that AMPK does not play a major role in the activation of GS at this time point during contractions, but if the exercise is continued, phosphorylation of GS at site 2 is increased. This phosphorylation of site 2 maintains GS activity at basal levels until the phosphorylation of sites 3a and 3b are decreased. In addition to its effects on AMPK, AICAR treatment of skeletal muscle increases GLUT4 recruitment in line with the degree of AMPK activation. The invention is useful in such applications by maintaining or enhancing AMPK phosphorylation and thus its activation, thereby increasing or maintaining/sustaining its downstream effects.

Metformin is a widely used drug in the treatment of type 2 diabetes that activates AMPK. Increased phosphorylation of AMPK at its catalytic T-loop residue, Thr172, in response to rising AMP levels thought to occur by a change in conformation of AMPK such that it is better phosphorylated by its upstream kinases. It is of importance, therefore, to understand the mechanisms involved in the dephosphorylation of AMPK, which are disclosed herein and include identification of the protein phosphatase(s) responsible for the dephosphorylation of AMPK, and the role(s) of these phosphatase(s) in the response of AMPK to phenformin and analogues thereof such as metformin.

PPM1F may preferentially associate with AMPKα1. Thus in some aspects the invention relates to the use of PPM1F in the dephosphorylation of AMPKα1. In one embodiment the invention relates to a method for purification of AMPKα1 comprising enriching for or purifying PPM1F.

PPM1E may preferentially associate with AMPKα2. In one embodiment the invention relates to a method for purification of AMPKα2 comprising enriching for or purifying PPM1E.

PPM1E and PPM1F may associate. Thus in some embodiments the invention may relate to a method for purifying PPM1E comprising enriching for or purifying PPM1F. Conversely, in some embodiments the invention may relate to a method for purifying PPM comprising enriching for or purifying PPM1E.

### Further Applications

Preferably uses of the invention are in vitro uses. More preferably uses of the invention in relation to metformin are in vitro uses. More preferably uses of the invention in relation to phenformin are in vitro uses.

Some embodiments of the invention may involve screening for agents which are activators of phosphatase activity in which case the step of comparing PPM phosphatase activity (e.g. determining whether it is lower in said first sample than in said second sample of the methods of the invention) is simply reversed so that an increase in activity in said first sample relative to said second sample identifies the agent as an activator. Particularly preferred are compounds such as candidate agents which are inhibitors of phosphatase activity. Suitably agents are inhibitors of phosphatase activity in cells (e.g. in cell lysates) as mentioned in the examples.

Effects on (such as inhibition of) phosphatase activity may be direct or indirect. Agents may bind or may not bind directly to the phosphatase or to AMPK. Agents may affect the interaction between the phosphatase and the AMPK e.g. by reducing or inhibiting the interaction or by encouraging dissociation. Thus the invention also relates to assays for agent(s) capable of affecting the interaction between AMPK and phosphatase. This might be by inhibition or promotion of co-immunoprecipitation in the presence of the agent(s) being assayed as set out in the examples, or by any other technique known to the skilled operator.

A key test is whether phosphatase activity is affected in vitro and optionally whether this effect is validated e.g. in cells (e.g. in cell lysates).

An aim of the invention is to identify inhibitors of the key phosphatases with a view to inhibiting those phosphatases in subject(s) in order to increase or maintain AMPK phosphorylation and thus activity which is useful in treatment of (e.g.) diabetes.

Suitably the phosphatase is PPM1E.

Suitably the phosphatase is PPM1F.

Suitably the phosphatase is PPM1E and PPM1F (e.g. a mixture, or an association or complex comprising both PPM and PPM1F).

### BRIEF DESCRIPTION OF THE FIGURES

FIG. 1 shows Analysis of type I protein phosphatases in HEK293 cells either untreated or treated with 10 mM phenformin for 1 hour. A Levels of phosphorylation of AMPK at Thr172 in 4 untreated and 4 treated samples. Anti-PP5 TPR domain antibody is used as a loading control. Molecular weights (kDa) are indicated to the left of the panels. B PP1 phosphorylase phosphatase activity in treated and untreated HEK293 cell lysates using phosphorylase a as substrate, in the presence of 4 nM okadaic acid. Data are mean±SEM for four samples measured in triplicate. C PP2A phosphorylase phosphatase activity in treated and untreated HEK293 cell lysates using phosphorylase a as substrate, in the presence of 200 nM I-2. Data are mean±SEM for four samples measured in triplicate.

FIG. 2 shows Mg²⁺ dependent okadaic acid resistant phosphatase (PPM phosphatase) activity in cell lysates either untreated or treated with 10 mM phenformin for 1 hour. A PPM phosphatase activity in treated and untreated HEK293 cell lysates using casein as substrate in the presence of 10 mM MgAc and 5 μM okadaic acid. Data are mean±SEM with five independent samples measured in duplicate. (p<0.001). B PPM phosphatase activity in treated and untreated HeLa cell lysates using casein as substrate in the presence of 10 mM MgAc and 5 μM okadaic acid. Data are mean±SEM with four independent samples measured in duplicate. (p<0.001).

FIG. 3 shows activity of recombinant PPM phosphatases after preincubation with 1 mM phenformin, using casein as substrate. Bacterially expressed PPM phosphatases were incubated with or without 1 mM phenformin for 15 minutes prior to assays being performed using ³²P casein as substrate in the presence of 10 mM MgAc and 5 μM okadaic acid. Assays were performed in triplicate.

FIG. 4 shows levels of PPM phosphatases in HEK293 cells either untreated or treated with phenformin. Immunoblotting of HEK293 cell lysates either untreated or treated with 10 mM phenformin for 1 hour. Cell lysates were immunoblotted using antibodies raised against the PPM enzymes indicated. Two representative samples are shown for each treatment. The predicted molecular weight in kDa are indicated in parentheses. Molecular masses of marker proteins in kDa are indicated to the left of the panels.

FIG. 5 shows activities of PPM phosphatases from treated and untreated HEK293 cell lysates after separation by FPLC. Lysates from control or phenformin-treated HEK293 cells were filtered through 0.45 and 0.22 μm filters and desalted using HiTrap desalting columns. An HR5/5 Source 15-Q column was utilised to separate proteins according to their net charge. Fractions were analysed for protein concentration and PP2C phosphatase activity in each fraction was measured using ³²P labelled casein as substrate, in the presence of 10 mM MgAc and 5 μM okadaic acid.

FIG. 6 shows activities of PPM phosphatases in HEK293 cells either untreated or treated with phenformin. PPM phosphatase activity in HEK293 cell lysates either untreated or treated with 10 mM phenformin for 1 hour. Individual isoforms were immunoprecipitated from control or phenformin-treated cell lysates and activity measured using ³²P labelled casein as substrate in the presence of 10 mM MgAc and 5 okadaic acid. Data are mean activities for assays performed in triplicate.

FIG. 7 shows domain structures of PPM1E and PPM1F. Conserved regions are shown in the hatched area; the black boxes represent a PP2C signature motif conserved in all family members (YFAVFDGHG) and the grey boxes indicate a cluster of acidic residues not found in PPM1.

FIG. 8 shows table of salt concentrations at which PPM phosphatases elute from an FPLC column. Immunoblotting was performed on fractions collected after HEK293 cell lysates were separated by FPLC. The approximate salt concentration at which each enzyme elutes is indicated together with the predicted and observed molecular masses in kDa of the bands detected.

FIG. 9 shows bar charts representing activities of PPM phosphatases in HEK293 cells either untreated or treated with phenformin (% scale of absolute data presented in FIG. 6).

FIG. 10 shows a bar chart. FIGS. 11 and 12 each show photographs of western blots.

The invention is now described by way of example. These examples are intended to be illustrative, and are not intended to limit the appended claims.

### EXAMPLES

### Example 1

### Effect of Biguanide on the Phosphorylation of AMPK

In order to determine whether the effects of biguanide (such as metformin or phenformin) might be mediated in part by effects on protein phosphatases, experiments were performed in order to investigate whether the metformin analogue phenformin affects the activity of protein phosphatases that may dephosphorylate AMPK. The effect of phenformin on protein phosphatases is described.

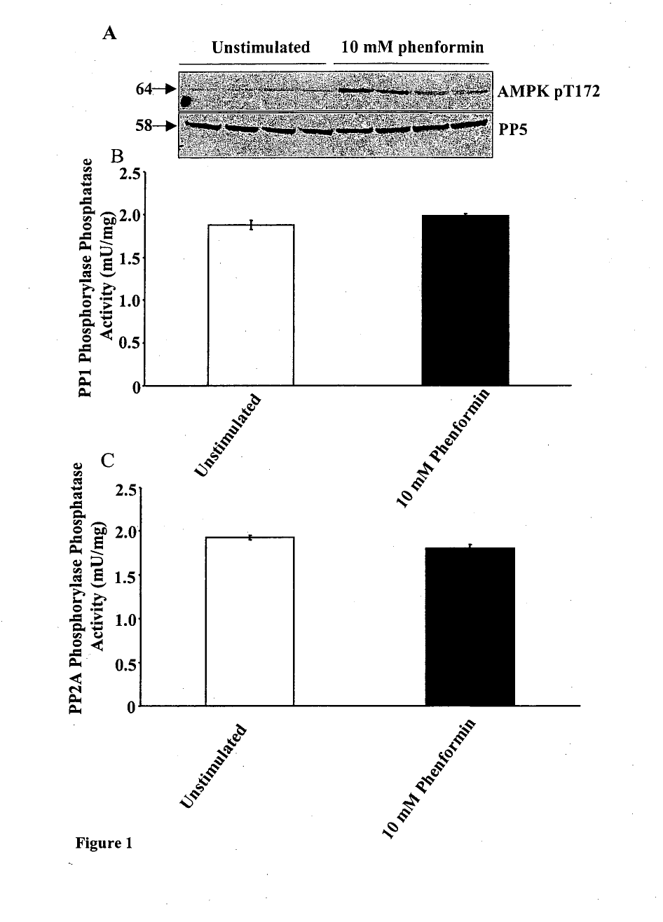

To study the effects of phenformin on AMPK activity, serum-starved HEK293 cells were stimulated with 10 mM phenformin for 1 hour at 37° C. Cells were lysed in lysis buffer containing 1 μM microcystin-LR to preserve the phosphorylation state of AMPK. Immunoblotting was performed using an antibody raised against a phosphopeptide corresponding to the area surrounding the catalytic Thr172 residue on AMPK. Increases in phosphorylation of AMPK at Thr172 could be observed after stimulation with phenformin. (FIG. 1A, upper panel).

### Example 2

### Effect of Biguanide on the Activity of Protein Phosphatases

To study the effects of biguanide on the activity of protein phosphatases, HEK293 cell lysates either untreated or treated with biguanide (10 mM phenformin in this example—as described above) were either immunoblotted or assayed for specific protein phosphatase activity by using combinations of phosphatase inhibitors and activators, as well as different phosphorylated substrates.

Since the activity of PP5 in HEK293 cells is low, the levels of PP5 were assessed by immunoblotting using an antibody against the TPR domains of the protein. No change in the levels of PP5 could be observed in response to phenformin (FIG. 1A, lower panel).

The activity of PP1 in response to phenformin was assessed using ³²P-labelled phosphorylase a as substrate with 4 mM okadaic acid included in the reaction in order to inhibit the activity of PP2A and EGTA (0.1-2 mM) to inhibit metal ion activated protein phosphatases. No change in PP1 phosphorylase phosphatase activity could be detected between unstimulated and 10 mM-phenformin stimulated cell lysates (FIG. 1B).

The activity of PP2A in response to phenformin was also assessed using ³²P labelled phosphorylase a as substrate, but for this assay 200 nM I-2 and EGTA (0.1-2 mM) to inhibit metal ion activated protein phosphatases was included to inhibit PP1. No change in the PP2A phosphorylase phosphatase activity could be detected between unstimulated and 10 mM-phenformin stimulated cell lysates (FIG. 1C).

### PPM Inhibitor Assay

The activity of PP2C (PPM1) and related members of the PPM family was assessed using ³²P-labelled casein as substrate with 5 μM okadaic acid included in the reaction to inhibit PP2A, and 10 mM magnesium acetate, which is known to be required for the activity of PP2C (Ingebritsen and Cohen, 1983 Science vol 221, pp 331-338; Ingebritsen et al., 1983 Eur J Biochem vol 132, pp 263-274).

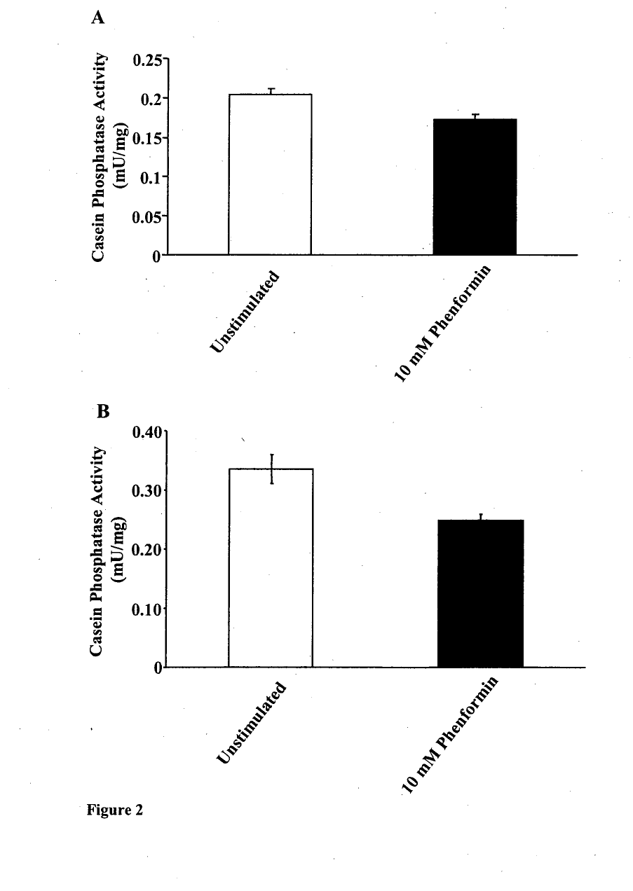

In HEK293 cells, the Mg²⁺ dependent, okadaic acid resistant casein phosphatase activity (hereafter termed PPM phosphatase activity) was decreased by ˜20% in cells treated with phenformin compared with untreated control cells (FIG. 2A), demonstrating an inhibitory effect of phenformin on this activity in cells i.e. in vivo.

In HeLa cells, which lack LKB1 (one of the upstream activators of AMPK), phenformin was unable to activate AMPK (Hawley et al., 2003 J. Biol vol 2, p28). To test whether phenformin is able to have similar effects in this cell line, PPM phosphatase activity in HeLa cells either untreated or treated with 10 mM phenformin was assessed. Again, a ˜20% decrease in PPM phosphatase activity could be observed in cells treated with phenformin (FIG. 2B). In this experiment, the activity of many PP2C isoforms is measured, and the 20% decrease observed may represent a more severe decrease in activity of only one such isoform.

The same experiments were conducted with 2 mM metformin instead of 10 mM phenformin. The same 20% decrease in PPM phosphatase activity was observed in cells treated with metformin.

Thus it is demonstrated for the first time that biguanides such as phenformin and metformin are inhibitors of PPM phosphatase activity, and appear to be specific inhibitors of said activity.

### Example 3

### Effect of Biguanide on the Activity of PPM Family Members

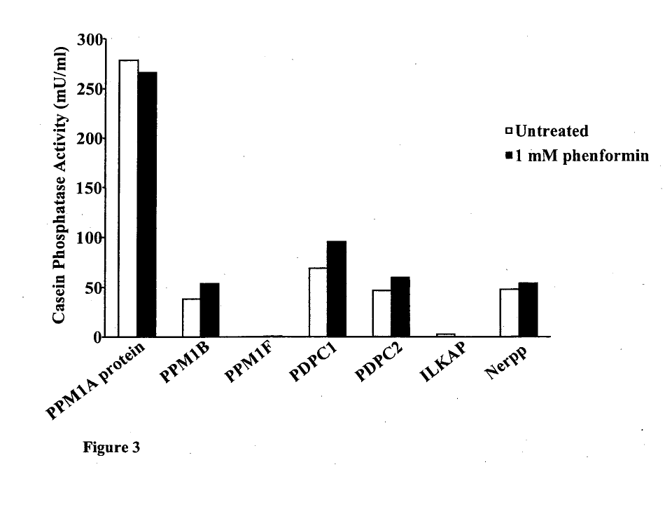

The PPM family of protein phosphatases comprises at least 16 structurally different isoforms with varying substrate specificities. To determine which of these phosphatases might have altered activity in response to biguanide, bacterially expressed PPM enzymes were tested. The PPM phosphatase activity associated with each enzyme was assessed after preincubation with buffer alone or with 1 mM biguanide (phenformin in this example). Of those enzymes which could be expressed in an active form (PPM1A, PPM1B, PDPC1, PDPC2, and Nerpp), no difference could be detected between control samples and those preincubated with phenformin. Bacterially expressed PPM1F and ILKAP showed no activity against phosphocasein under these assay conditions (FIG. 3). PPM1D, PPM1E and PPM1G were not tested in this experiment.

### Example 4

### Effect of Biguanide on the Level of PPM Family Members

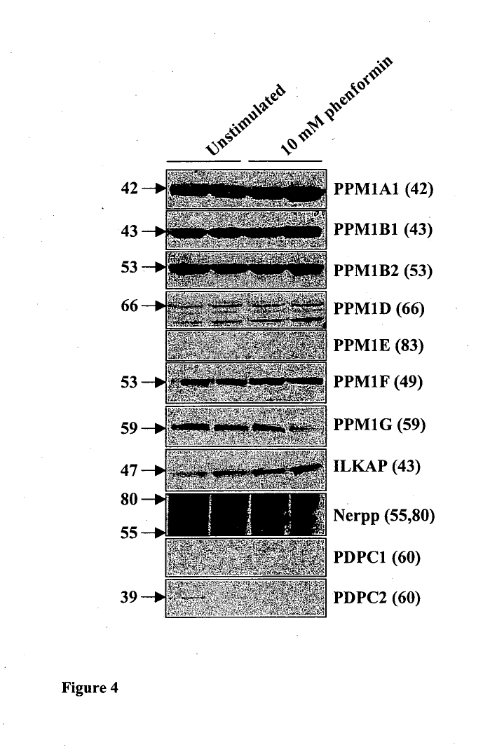

To determine whether biguanide was able to have any effect on the levels of any of the PPM phosphatases, immunoblotting was performed using antibodies. Antibodies raised against PPM1A, PPM1B1, PPM1B2, PPM1D, PPM1F, PPM1G and ILKAP produced bands close to the predicted molecular weights of the enzymes, although PPM1D also produced a number of other bands. No differences in the levels of any of these enzymes could be detected between untreated cells and those stimulated with biguanide (phenformin in this example) (FIG. 4). Antibodies against PPM1E and PDPC1 did not produce any signal, whilst PDPC2 and Nerpp gave rise to a faint band at the wrong molecular size and general background staining respectively.

### Example 5

### PPM Activities in Treated and Untreated Cell Lysates Separated by FPLC

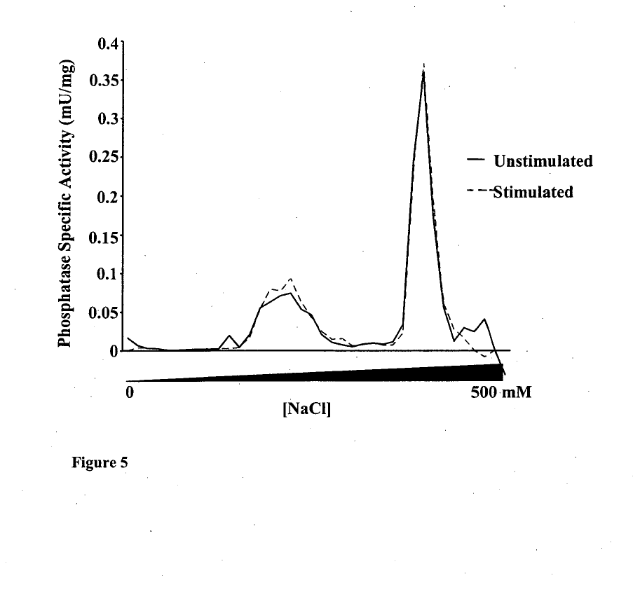

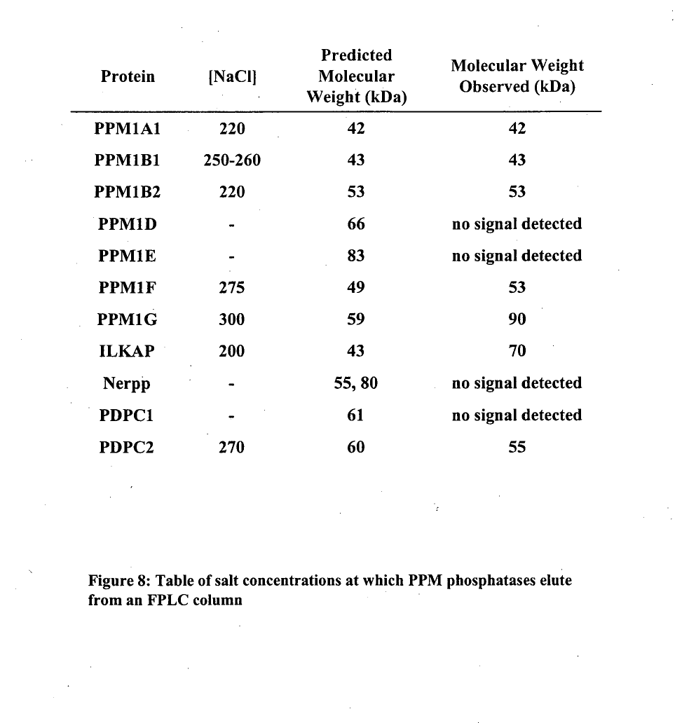

In order to identify the precise PPM phosphatases inhibited by biguanide such as phenformin, HEK293 cell lysates from treated and phenformin-treated cells were filtered and desalted before being separated according to net charge by FPLC. Fractions of 500 μl were collected and total PPM phosphatase activity in each fraction was measured. Some slight differences in protein phosphatase activity were detectable between untreated and phenformin-treated samples eluting at around 150 mM and 500 mM NaCl from the FPLC column (FIG. 5). Immunoblotting of fractions from the FPLC column identified some PPM phosphatases and the salt concentrations at which they elute (table—see FIG. 8), although the largest peak visible in FIG. 5 was not identified by this method.

In this experiment, antibodies against PPM1G and ILKAP identified bands of the incorrect size, whilst PPM1D, PPM1E, Nerpp and PDPC1 could not be detected. It is possible that the PPM activity that is inhibited by phenformin elutes from the FPLC column at around either 150 or 500 mM NaCl (FIG. 5). Without wishing to be bound by theory, the results do not exclude the possibility that a small molecule bound to one of the PPM isoforms is washed off in the column thus preventing detection of the change in activity observed previously.

### Example 6

### Effect of Biguanide on the Activity of Specific PPM Enzymes

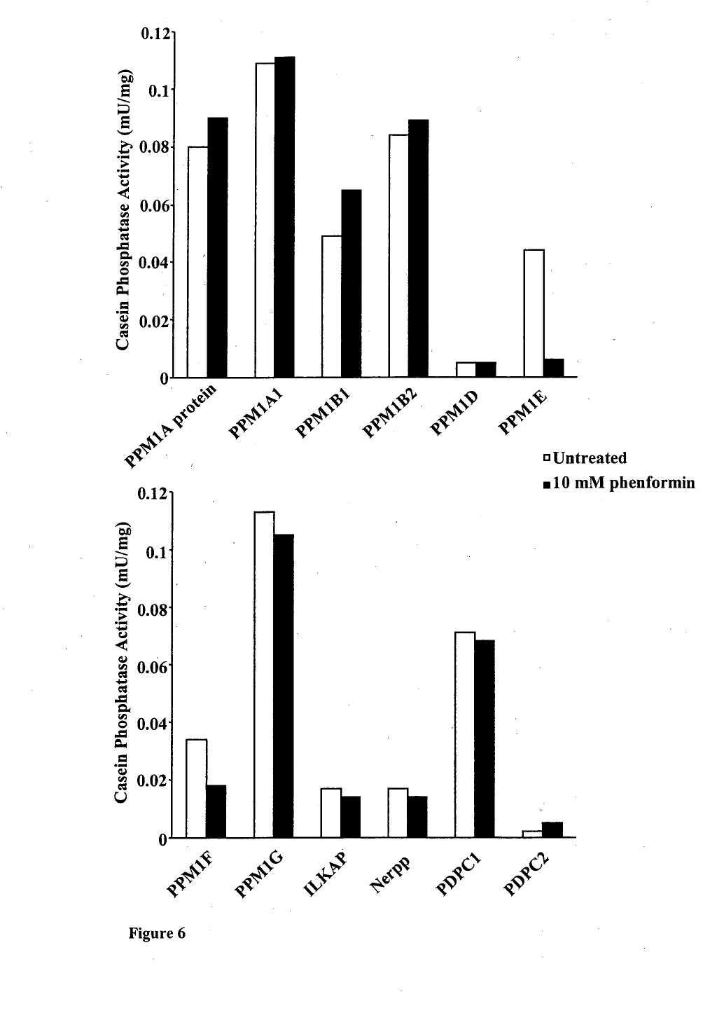

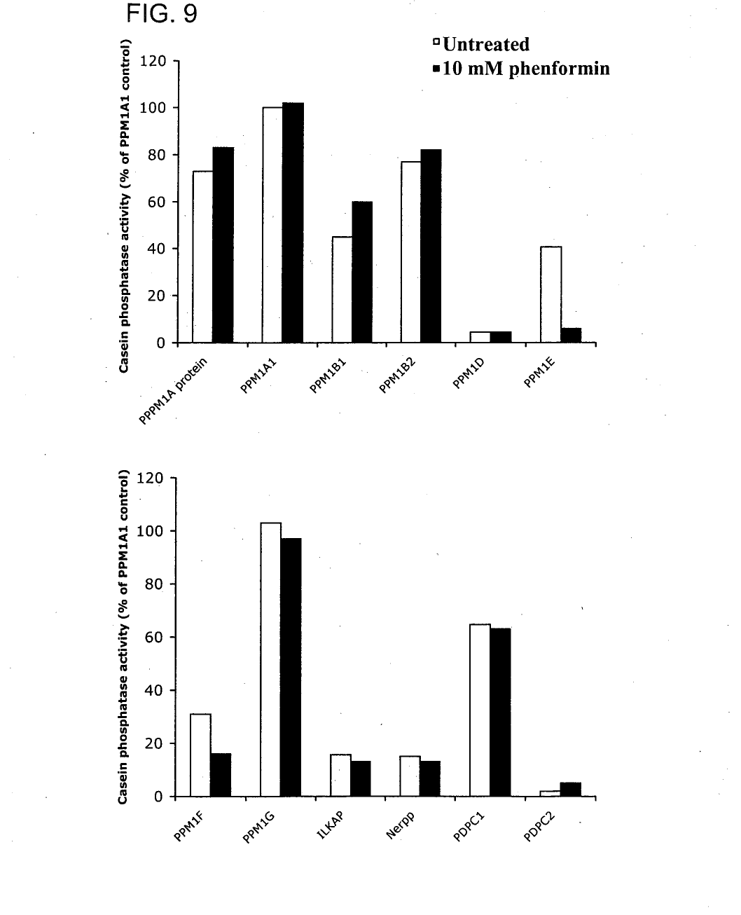

To determine with greater certainty which PPM phosphatases are affected by biguanide, the PPM phosphatase activity associated with each of the PPM enzymes was assessed. In this example, the biguanides are metformin and phenformin. Immunoprecipitation of the PPM phosphatases was performed using peptide antibodies from HEK293 cell lysates which were untreated or had been treated with 10 mM phenformin (FIG. 6/FIG. 9; with reference to FIG. 9, the scale is a percentage scale for ease of comparison since the absolute values in FIG. 6 can vary depending on non-substantive experimental factors such as the level of incorporation of the 32P label). No change could be detected in the PPM phosphatases associated with PPM1A1, PPM1B1, PPM1B2, PPM1D, PPM1G, ILKAP, Nerpp, PDPC1 or PDPC2. Interestingly, however, the activity of PPM 1E was almost completely ablated after treatment with phenformin, whilst the activity of the closely related PPM1F was decreased by ˜40% (FIG. 6). Following immunoprecipitation and immunoblotting of the PPM1E immunoprecipitate from untreated HEK293 cells, no signal could be detected using an antibody directed against PPM1E.

### Example 7

### Biguanide has No Effect on PPP Phosphatases, but Inhibits PPM Phosphatases

Biguanide such as phenformin causes activation of AMPK in HEK293 cells by increasing the phosphorylation of the α subunit at the Thr172 residue. This effect is decreased in HeLa cells since these cells lack LKB1, one of the upstream kinases of AMPK. The activity of LKB1 itself is not affected by phenformin and so it was therefore of key importance to assess whether phenformin might be able to affect the activity of the protein phosphatase responsible for the dephosphorylation of AMPK. We considered that a protein phosphatase which is insensitive to okadaic acid is responsible for the dephosphorylation of AMPK in intact cells and evidence has been reported to suggest that the metal-ion dependent PP2C (PPM1) might be responsible for dephosphorylation of AMPK in-vivo, although in-vitro, both PP2A and PP2Cα (PPM1A) are able to perform this role.

Stimulating HEK293 cells with phenformin had no effect on the activity of PP1 or PP2A as measured in an in-vitro phosphatase assay or on the levels of PP5. However, in both HEK293 cells and HeLa cells, phenformin is able to inhibit PPM phosphatase activity by around 20%. Together with the evidence that a PPM enzyme may be the primary phosphatase responsible for dephosphorylation of AMPK, this raised the intriguing possibility that phenformin might be acting to inhibit the activity of a PPM enzyme and thus increase AMPK activity. Since the exact mechanism(s) by which phenformin activates AMPK is unknown in the art, inhibition of a protein phosphatase activity represents a novel mechanism for the activation of AMPK by anti-diabetic drugs. Thus it is demonstrated that PPM phosphatases are targets for therapeutic intervention in such disorders.

### Example 8

### Effect of biguanide on PPM enzymes

A number of different isoforms of PPM enzymes exist and the casein phosphatase activity of a number of bacterially expressed PPM phosphatases was unaffected by preincubation with biguanide such as phenformin. PPM1A, PPM1B, PPM1F, PDPC1, PDPC2, ILKAP and Nerpp were able to be expressed and purified. Casein phosphatase activity was detected in the preparations of PPM1A1, PPM1B, PDPC1, PDPC2 and Nerpp, but this was unaffected by preincubation with 1 mM concentrations of phenformin, arguing against a role for phenformin in directly binding and inhibiting these enzymes. No conclusions can be drawn from this specific experiment with regard to PPM1F or ILKAP since these purified enzymes displayed no activity against phosphocasein.

Specific antibodies against individual PPM enzymes did not detect any changes in the level of many of the PPM phosphatases. These immunoblotting experiments argue against phenformin acting to alter the level of PPM1A1, PPM1B1, PPM1B2, PPM1D, PPM1F, PPM1G or ILKAP. These specific immunoblotting experiments were unable to determine whether the levels of PPM1E, PDPC1, PDPC2 or Nerpp were affected by phenformin.

Separation of PPM activities by FPLC resulted in two major peaks of phosphatase activity. It has been suggested that two peaks of activity from skeletal muscle lysates represented PP2Cα (PPM1A) and PP2Cβ (PPM1B), although in HEK293 cells each peak contains more than one PPM phosphatase activity.

Use of specific antibodies against PPM phosphatases to immunoprecipitate and assay the casein phosphatase activity associated with each of the PPM enzymes was performed. Interestingly, the activity of PPM1E was almost completely ablated in cells treated with the biguanide phenformin or metformin and the activity of PPM was decreased to ˜60% of the levels of untreated cells. PPM and PPM are two closely related enzymes, sharing 66% similarity in the core phosphatase domain and homologous flanking sequences and each containing a fused PIX-binding domain. PPM1E is involved in the dephosphorylation of the p21-activated kinase PAK. PPM1F has been shown to be able to dephosphorylate the catalytic Thr286 residue in autophosphorylated calcium/calmodulin dependent protein kinase II (CaMKII) as well as CaMKI and CaMKIV.

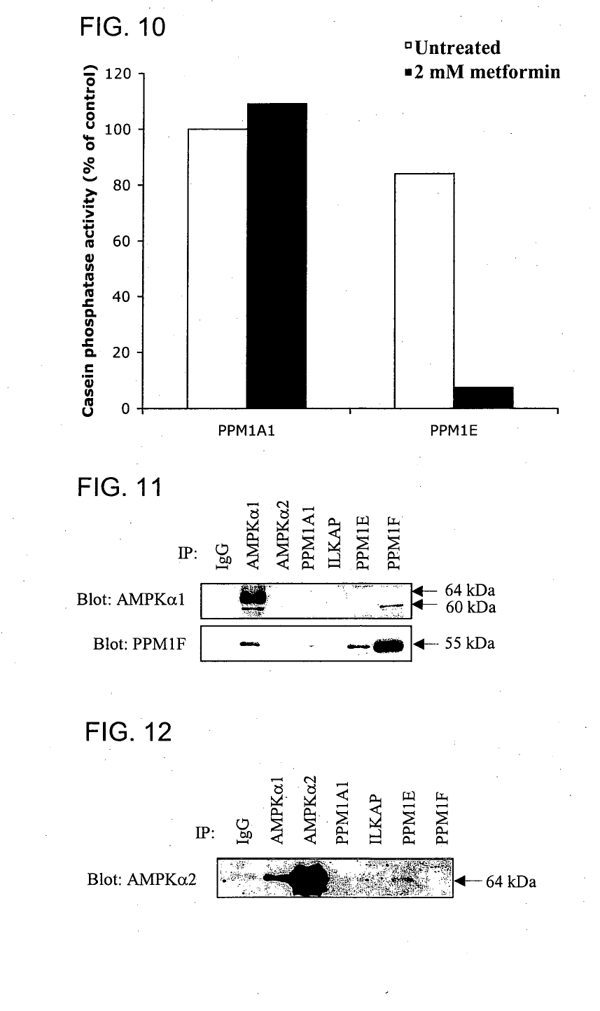

FIG. 10 shows activities of the PPM phosphatases PPM1A1 and PPM1E in HEK293 cells treated or untreated with 2 mM metformin. HEK293 cells were treated with 2 mM metformin for 10 minutes prior to lysis. The PPM1A1 and PPM1E isoforms were immunoprecipitated in triplicate from untreated or metformin-treated HEK293 cell lysates and activity measured using 32P labelled casein as a substrate in the presence of 10 mM magnesium acetate and 5 μM okadaic acid. Control immunoprecipitations were preformed in triplicate for each lysate using pre-immune antibody and subtracted from the activity calculated for the PPM phosphatase immunoprecipitations. The activities are presented relative to untreated PM1A1. Substantial specific knockdown of PPM1E activity by Metformin treatment is demonstrated.

Within the human kinome (http://www.kinase.com/human/kinome), protein kinases are grouped into a number of different kinase families according to the sequence similarity of their catalytic domains, the domain structure outside of the catalytic domains, their known biological functions and comparison of their classification in other species. Both AMPK and CaMKK families of protein kinases are contained within the CaMK superfamily. The regulation of both AMPK and CaMK are intriguingly similar; both enzymes require allosteric activation by their appropriate ligands (AMP for AMPK and Ca²⁺/calmodulin for CaMKK) followed by phosphorylation of a threonine residue within the activation loop.

The results described here have at least two possible meanings. Firstly, given the structural similarities between CaMKs and their upstream kinases CaMKKα and CaMKKβ, PPM1E and PPM1F might act to dephosphorylate CaMKK isoforms, thus decreasing their activity and accordingly decreasing the activity of AMPK. Secondly, given the conserved mechanism of activation of both CaMKs and AMPK discussed above and the fact that both AMPK and CaMKs reside within the same protein kinase superfamily, PPM1E and PPM1F might be acting to dephosphorylate AMPK directly.

In either scenario, it is demonstrated that biguanide such as phenformin is able to inhibit protein phosphatase activity that has been implicated in the dephosphorylation of AMPK. Thus it is demonstrated that PPM phosphatase is a valid therapeutic target for modulation of AMPK activity and that inhibitors of PPM phosphatase are excellent candidate therapeutics for same.

### Example 9

### The Effect of Biguanide on Members of the PPM Family Protein Phosphatases

The data presented here are the results of studies to identify both the protein phosphatase responsible for dephosphorylation of AMPK and a target for antidiabetic drugs such as metformin and phenformin. One of the mechanisms by which these compounds lower blood glucose levels is by increasing the activity of AMPK; an understanding of this mechanism of action has been absent from the art. We disclose that one or more members of the PPM family of protein serine/threonine phosphatases is involved in the increasing activity of AMPK in response to biguanide compounds.

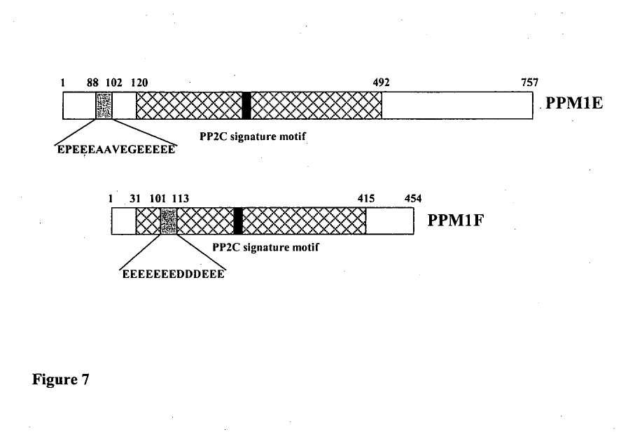

That the PPM family protein phosphatases PPM1E and PPM1F may be acting to dephosphorylate AMPK in response to metformin/phenformin is a significant advance. AMPKα₂-containing complexes have a greater dependence on AMP and are enriched in the nucleus compared with AMPKα₁-containing complexes where they are postulated to play a role in gene transcription. Exercise induced translocation of AMPK α₂ to the nucleus has been seen, but the mechanism by which this occurred was unclear in the art. PPM1E and PPM1F share 64% homology in their phosphatase domain but PPM1E has large regions without homology in both the N- and C-termini (FIG. 7). Two nuclear localisation signals in the C-terminus of PPM1E have been identified as well as a cluster of basic residues essential for their function. PPM1F, CaMKI, CaMKII and CaMKKβ localise specifically to the cytoplasm, whilst PPM1E, CaMKIV and CaMKKα localise specifically to the nucleus, suggesting that two CaMK regulatory systems occur within cells, and that PPM1E and PPM1F might play complementary roles within the cell. PPM1E is most abundantly expressed in brain in immunoblotting experiments using an antibody raised against the C-terminal residues of the protein, similar to that used in this study. Without wishing to be bound by theory, it is possible therefore that the expression of the protein is low in other tissues and may explain the fact that no protein can be detected by immunoblotting in these specific experiments.

The majority of the PPM1E present in the brain exists in a truncated form that is most abundant in the cytoplasm, raising the possibility that PPM1E might be able to dephosphorylate both cytosolic and nuclear CaMK family protein kinases.

Metformin is known to lower glucose and lipids by both decreasing hepatic glucose production and increasing skeletal muscle glucose uptake, and AMPK was first postulated as a potential mediator of the effects of the drug, but the mechanism by which metformin activates AMPK has long been an enigma in the art. Metformin has been shown to be able to inhibit complex I of the respiratory chain and therefore impairs mitochondrial function and cell respiration, thereby inhibiting hepatic glucose production and increasing glucose utilisation. Metformin inhibits glucose production primarily by inhibition of hepatic glycogenolysis. There is some controversy over the involvement of metformin in increasing the cellular AMP:ATP ratio. It has been reported that metformin activates AMPK via an adenine nucleotide-independent mechanism but another report states that incubation of a range of cell types with the more potent biguanide phenformin increases the AMP:ATP ratio. It seems likely that the effects of metformin on the AMP:ATP ratio, which occur over a greater time period and with less efficacy, are more difficult to detect. Metformin inhibition of PPM1E and/or PPM1F activities and whether this inhibition is dependent on changes in the AMP:ATP ratio may be important.

Since calcium and calmodulin regulate the activity of the CaMK family and therefore AMPK, the possibility exists that calcium and calmodulin might also have an effect on PPM1E and PPM1F. Under the assay conditions used in the experiments shown here, no calcium ions should be present in the reaction mixture. Further experiments in which calcium and/or EGTA was added to the reaction mixture may be used to assess whether calcium had any effect on the activities of these enzymes and their inhibition by metformin/phenformin. In addition, it may be useful to utilise more specific substrates than phosphocasein in order to understand whether the phosphatases PPM1E and PPM1F are able to dephosphorylate specific members of the CaMK protein kinase superfamily. It may also help to assess the effects of metformin/phenformin directly on the activity of PPM1E after immunoprecipitation of the enzyme from unstimulated cell extracts.

Studies using short interfering RNA (siRNA) are useful in demonstrating whether knockdown of the levels of PPM or PPM has profound effects on AMPK activity and whether metformin/phenformin activates AMPK in the absence of the phosphatase. Such studies find application in assessing the relative contributions of each of these enzymes to the dephosphorylation of their substrates in vivo.

### Example 10

### Assay/Screen

A method for identifying a candidate agent for use in a medicament for diabetes or obesity is demonstrated. The method comprises providing a candidate inhibitor of PPM phosphatase, providing a first and a second sample comprising PPM phosphatase, contacting said candidate inhibitor with said first sample comprising PPM phosphatase, and assaying said first and second samples for PPM phosphatase activity. In this example, the PPM phosphatase is PPM1E.

PPM1E is immunopelleted with antibodies raised the amino acid sequence KTHDIPCPDLPWSY.

The phosphatase activity in the immunopellet is measured in a protein phosphatase assay using 32P-labelled casein as substrate in the presence of 10 mM Mg2+ or Mn2+ ions and 5 μM okadaic acid. In this example, the assay is conducted as follows:

The washed immune pellet is resuspended in 10 μl buffer B. Protein phosphatases are assayed at 30° C. in a volume of 30 μl, with 1 μM-10 μM 32P-labelled substrate. In this example the 32P-labelled casein substrate is partially hydrolysed bovine milk casein (Sigma, Poole UK) labelled with [γ32P]ATP using the catalytic subunit of protein kinase A.

The 32P-labelled substrate and phosphatase inhibitors/activators are diluted separately in buffer C. The assay is performed by mixing the immunopellet in 10 μl buffer B with 10 μl of the inhibitor/activator or buffer C and incubating the mixture at 30° C. for 10 min. The assay is started by the addition of 10 μl of 32P-labelled substrate. The assay is then incubated for a further time (15 min) with shaking at 1200 rpm at 30° C. and stopped by the addition of 100 μl 20% (w/v) trichloroacetic acid. The mixture is vortexed briefly and centrifuged at 14,000×g for 5 minutes. 100 μl of supernatant is recovered and the 32P released is measured by Cerenkov counting on a Wallac 1409 liquid scintillation counter.

| | |
|---|---|
| Buffer A | 50 mM Tris-HCl pH 7.5, 0.1 mM EGTA, 0.1% (v/v) 2- |
| | mercaptoethanol. |
| Buffer B | Buffer A containing 1 mg/ml BSA. |
| Buffer C | Buffer A containing 0.01% (v/v) Brij-35. |

The PPM phosphatase activity is then compared in said first and second samples; when the activity is lower in said first sample than in said second sample then said candidate inhibitor is identified as a candidate agent for use in a medicament for diabetes or obesity.

### Example 11

### Validation of PPMs as Targets

In order to further characterise the relationships between elements in the signalling pathways discussed herein, and in order to provide further ways of validating candidate compounds identified by the method(s) of the invention, we investigated the molecular interactions.

AMPKα1, AMPKα2, several PPMs and control IgG covalently coupled to Sepharose beads were used to immunoabsorb their respective antigens from HEK293 cell lysates. Results are shown in FIGS. 11 and 12. The immuno-pellets (IPs) were washed, dissolved in 20 μl SDS gel loading buffer and analysed by SDS-PAGE and immunoblotting with the indicated antibodies. Sizes of bands (in kilodaltons) are indicated on the right-hand side.

### Materials and Methods

HEK293 cells were lysed in 50 mM Tris-HCl pH 7.5, 1 mM EGTA, 1 mM EDTA, 1% (v/v) Igepal CA-630 (NP-40 substitute), 1 mM sodium orthovanadate, 10 mM sodium-β-glycerophosphate, 50 mM sodium fluoride, 5 mM sodium pyrophosphate, 0.27 M sucrose, 5 mM N-ethylmaleimide, “Complete” protease inhibitor cocktail (one tablet/50 ml). Lysates were diluted approx 5-fold to 1 mg/ml in 50 mM Tris-HCl pH 7.5, 150 mM sodium chloride, “Complete” protease inhibitor cocktail (one tablet/50 ml). Immunoadsoroption was performed using diluted cell lysates containing 100 μg protein with addition of antibody-Sepharose (prepared from 10 μl Sepharose beads and 10 μg antibody). Following immunoabsorption for 3 hours at 4° C., immuno-pellets were centrifuged at 13,000×g for 5 min and washed twice in ice-cold 50 mM Tris-HCl pH 7.5, 150 mM sodium chloride.

Antibodies were affinity purified against their respective human antigens, used for blotting at concentrations of 1 μg/ml. and detected by enhanced chemiluminescence. Anti-AMPKα1 raised to AMPKα1 (344-CTSPPDSFLDDHHLTR-358) and anti-AMPKα2 raised to AMPKα2 (352-CMDDSAMHIPPGLKPH-366) were from Prof. D. G. Hardie (University of Dundee). Anti-PPM1A1 raised to PPM1A1 (369-KNDDTDSTSTDDMW-382), anti-ILKAP raised to ILKAP (373-KAVQRGSADNVTVMV-387), anti-PPM1E raised to PPM1E (728-RSSLPWRQNSWK-739) and anti-PPM1F raised to PPM1F (439-LPSSLPEPETQAPPRS-454), anti-PPM1K raised to PPM1K (359-FSFSRSFASSGRWA-372) were prepared in the Division of Signal Transduction Therapy, managed by Dr Hilary McLauchlan and Dr James Hastie (University of Dundee). Anti-PHUPP-L was from Novus Biologicals (Littleton, Colo.). The control IgG was a pre-immune IgG fraction from serum of sheep in which an immune response had not been raised.

FIG. 11 shows that endogenous PPM1F is present in immuno-pellets of endogenous AMPKα1 (lane 2) but not AMPKα2 (lane 3). The reciprocal immuno-pellets show the presence of AMPKα1 (lane 7) but not AMPKα2 in PPM1F immuno-pellets. Note that in some circumstances PPM1E and PPM1F may associate leading to the presence of some PPM1F in PPM1E immunopellets (lane 6). Immuno-pellets of endogenous PPM1E showed the presence of a band of endogenous AMPKα2 but not AMPKα1 (FIG. 12).

Summary of the association of several PPM phosphatases with
AMPKα1 and AMPKα2:

| | Mass kDa | | |
|---|---|---|---|
| | predicted (observed | Association with | Association with |
| Phosphatase | on SDS-PAGE) | AMPKα1 | AMPKα2 |
| PPM1A1 | 42 (42) | No | No |
| PPM1B1 | 43 (43) | No | No |
| PPM1B2 | 53 (53) | No | No |
| PDPC2 | 60 (39) | No | No |
| PPM1E | 84 (84) | No | Weak interaction |
| | | | observed |
| PPM1F | 50 (61) | Good interaction | No |
| | | observed | |
| PPM1G | 59 (59) | No | No |
| ILKAP | 43 (47) | No | No |
| PHLPP-L | 147 (~200) | No | No |

The table shows the presence or absence of the indicated PPM phosphatase in the IPs of AMPKα1 or AMPKα2.

The data in the above table list the PPMs tested in experiments that were not present in the AMPKalpha1 IP. In addition there are data which suggest PPM1F can be found in the AMPKalpha1 IP.

The signal obtained for PPM1E in an AMPKalpha2 IP is very low. Without wishing to be bound by theory, the PPM1E antibody may not IP well and therefore the AMPKalpha2 in the IP may be low (as detectable by immunoblotting) for this reason. In any case it should be noted that the IPs presented in FIGS. 11 and 12 are of endogenous proteins (rather than overexpressed proteins), which are technically demanding to perform. However, data with endogenous proteins as presented provide scientifically very strong support for the interaction and thus validate the targets and methods disclosed herein.

### Example 12

### In Vitro PPM Assays

A method for identifying a candidate agent for use in a medicament for diabetes or obesity is demonstrated. The method comprises providing a candidate inhibitor of PPM phosphatase, providing a first and a second sample comprising PPM phosphatase, contacting said candidate inhibitor with said first sample comprising PPM phosphatase, and assaying said first and second samples for PPM phosphatase activity. In this example, the PPM phosphatase is PPM1E.

In this example PPM1A1 is produced in E. coli as described in Davis et al. 1995 (Davies, S. P., Helps, N. R., Cohen, P. T. W. and Hardie, D. G. (1995) FEBS Lett. 377, 421-425.*5*-AMP inhibits dephosphorylation, as well as promoting phosphorylation of the AMP-activated protein kinase; studies using bacterially expressed human protein phosphatase-2Calpha and homogeneous native bovine protein phosphatase-2AC.*).

In more detail, GST-PPM1(230-755) {carboxy-terminal two-thirds} and GST-PPM1F(2-454) {full-length} were expressed in E. coli in the absence or presence of Mn2⁺, as taught in Davies et al (ibid.) except that the expression was induced at 15° C. PPM phosphatase activity is determined by the release of [³²P]-orthophosphate from a glutathionine-S-transferase-peptide substrate GST-(GGGGRRAT[p]VA)₃ substrate in the presence of okadaic acid to inhibit PP1 and PP2A like activities.

GST-(GGGGRRAT[p]VA)₃ phosphatase substrate is prepared by phosphorylation with protein kinase A (PKA). 2 mg of bacterially expressed GST-(GGGGRRATVA)₃ is incubated with 1-2 mU PKA overnight at 30° C. with gentle shaking in a buffer consisting of 50 mM Tris-HCl pH 7.0, 0.1 mM EGTA, 10% glycerol, 10 mM magnesium acetate, 0.1% (v/v) 2-mercaptoethanol, 0.1 mM [gamma³²P]ATP.

Labelled GST-(GGGGRRAT[p]VA)₃ is separated from unincorporated radionucleotide by column chromatography on a glutathione-Sepharose column, eluting in 50 mM Tris-HCl pH 7.0, 0.1 mM EGTA, 10% glycerol, 10 mM magnesium acetate, 0.1% (v/v) 2-mercaptoethanol, 20 mM glutathione. GST-(GGGGRRAT[p]VA)₃ is diluted to 1-4 μM prior to addition during assays.

Phosphatases are assayed in a total volume of 30 p. 1 at 30° C. for ten minutes, with constant shaking. The first and second samples of the assay contained the phosphatase diluted in 20 μl buffer, together with the candidate inhibitor in the first sample, which first and second samples are then incubated at 30° C. for two minutes prior to addition of 10 μl 32P-labelled GST-(GGGGRRAT[p]VA)₃ phosphatase substrate. After ten minutes, reactions are terminated by addition of 100 μl 20% trichloracetic acid. Tubes are then vortexed for a further minute to ensure complete mixing and centrifuged at 16,000×g for 5 minutes at room temperature. 100 μl supernatant is removed from each reaction into a new Eppendorf tube and counted by Cerenkov counting in a liquid scintillation counter.

Acid-molybdate extractions reveal very little contaminating protease activity.

Phosphatase assay composition (final concentrations):

### 50 mM Tris-HCl pH 7.0

### 0.1 mM EGTA

10 mM magnesium acetate
2 mM manganese (II) chloride
5 μM okadaic acid
0.1% (v/v) 2-mercaptoethanol

Mass of phosphatase used in the assays is typically 10 ng-10 μg depending on the preparation.

The PPM phosphatase activity is then compared in said first and second samples; when the activity is lower in said first sample than in said second sample then said candidate inhibitor is identified as a candidate agent for use in a medicament for diabetes or obesity.

### Example 13

### High Throughput Assays

The assays of examples 10 or 12 may be conducted in high throughput format. In this example the assays are read out in non-radioactive form. Other details are as example 10 or 12 except as follows:

The release of phosphate after the 10 minute assay (before the addition of trichloracetic acid) is measured by the addition of 150 μl of 1 N HCl containing 10 mg/ml ammonium molybdate and 0.38 mg/ml malachite green to a 100 μl assay. After 20 minutes at room temperature (18-25° C.), the absorbance is measured at 620 nm. In this format, the 250 μl samples' absorbance is read out on a plate reader.

The PPM phosphatase activity is then compared in said first and second samples; when the activity is lower in said first sample than in said second sample then said candidate inhibitor is identified as a candidate agent for use in a medicament for diabetes or obesity.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A method for identifying a candidate agent for use in a medicament for diabetes or obesity said method comprising
a. providing a candidate inhibitor of PPM phosphatase,
b. providing a first and a second sample comprising PPM phosphatase,
c. contacting said candidate inhibitor with said first sample comprising PPM phosphatase, and
d. assaying said first and second samples for PPM phosphatase activity, wherein said PPM phosphatase is selected from the group consisting of PPM1E, PPM1F, PPM1J, PPM1K, PPM1L and PPM1M;
wherein if the PPM phosphatase activity is lower in said first sample than in said second sample then said candidate inhibitor is identified as a candidate agent for use in a medicament for diabetes or obesity.

2. The method according to claim 1 wherein the PPM phosphatase is at least one of PPM1E and PPM1F.

3. The method according to claim 1 wherein said disorder is diabetes.

4. The method according to claim 3 wherein said diabetes is type II diabetes.

5. The method according to claim 1 wherein said candidate inhibitor is a metformin or phenformin analogue or derivative.

6. A method of treatment or prevention of diabetes or obesity, said method comprising inhibiting PPM phosphatase in a subject.

7. The method according to claim 6 wherein said PPM phosphatase is selected from the group consisting of PPM1E, PPM1F, PPM1J, PPM1K, PPM1L and PPM1M.

8. The method according to claim 6 wherein said PPM phosphatase is PPM1E.

9. The method according to claim 6 wherein said PPM phosphatase is PPM1F.

10. A method of inhibiting a PPM type protein phosphatase, said method comprising contacting the PPM type protein phosphatase with metformin, phenformin, an analogue of metformin, an analogue of phenformin or a combination of the foregoing.

11. The method of claim 10 wherein said PPM type protein phosphatase is PPM1E or PPM1F.

12. The method of claim 10 wherein said PPM type protein phosphatase is selected from the group consisting of PPM1E, PPM1F, PPM1J, PPM1K, PPM1L and PPM1M.

13. A composition for use as a PPM phosphatase inhibitor, said composition comprising metformin, phenformin or a combination of the foregoing.

14. The method of claim 10 wherein the phosphorylation state of AMPK is maintained or enhanced.

15. (canceled)

16. The method of claim 10 wherein p21-activated kinase (PAK) is activated.

17. The method of claim 10 wherein the phosphorylation state of Ca2⁺/Calmodulin dependent kinase II (CaMKII) is maintained or enhanced.

18. A medicament comprising an agent identified by the method of claim 1, wherein said medicament is a medicament for diabetes or obesity.

19. (canceled)

20. (canceled)

21. (canceled)

22. The method of claim 6 where the compound is identified by the method comprising:
a. providing a candidate inhibitor of PPM phosphatase,
b. providing a first and a second sample comprising PPM phosphatase,
c. contacting said candidate inhibitor with said first sample comprising PPM phosphatase, and
d. assaying said first and second samples for PPM phosphatase activity,
wherein said PPM phosphatase is selected from the group consisting of PPM1E, PPM1F, PPM1J, PPM1K, PPM1L and PPM1M;
wherein if the PPM phosphatase activity is lower in said first sample than in said second sample then said candidate inhibitor is identified as a candidate agent for use in a medicament for diabetes or obesity.
